SEQUENCE LISTING (159 sequences)

SEQ ID NO: 1 - DNA, 51 bp, Artificial Sequence
  Synthetic
  misc_feature (34)..(39): n is a, c, g, t or u
gatcggaagagcacacgtctgaactccagtcacnnnnnnatctcgtatgcc

SEQ ID NO: 2 - DNA, 58 bp, Artificial Sequence
  Synthetic
aatgatacggcgaccaccgagatctacactctttccctacacgacgctcttccgatct

SEQ ID NO: 3 - DNA, 58 bp, Artificial Sequence; identical to SEQ ID NO: 2
  Synthetic

SEQ ID NO: 4 - DNA, 57 bp, Artificial Sequence
  Synthetic
gatcggaagagcacacgtctgaactccagtcacatctcgtatgccgtcttctgcttg

SEQ ID NO: 5 - DNA, 57 bp, Artificial Sequence; identical to SEQ ID NO: 4
  Synthetic

SEQ ID NO: 6 - DNA, 58 bp, Artificial Sequence; identical to SEQ ID NO: 2
  Synthetic

SEQ ID NO: 7 - DNA, 48 bp, Artificial Sequence
  Synthetic
gctcttccgatctgggttcctctgtgattccccgcagaggatttcgtg

SEQ ID NO: 8 - DNA, 48 bp, Artificial Sequence
  Synthetic
gctcttccgatctgggttcctctgtgattcctcgcagaggatttcgtg

SEQ ID NO: 9 - DNA, 42 bp, Artificial Sequence
  Synthetic
gctcttccgatctgggttcccaagggcgacgccgctcacctc

SEQ ID NO: 10 - DNA, 42 bp, Artificial Sequence
  Synthetic
gctcttccgatctgggttcccaagggcgacgacgctcacctc

SEQ ID NO: 11 - DNA, 40 bp, Artificial Sequence
  Synthetic
gctcttccgatctgggttcctctcccttgccttcttttga

SEQ ID NO: 12 - DNA, 40 bp, Artificial Sequence
  Synthetic
gctcttccgatctgggttcccaaggttctccttcctggag

SEQ ID NO: 13 - DNA, 41 bp, Artificial Sequence
  Synthetic
gctcttccgatctgggttcctctcctgtctgttactgccct

SEQ ID NO: 14 - DNA, 42 bp, Artificial Sequence
  Synthetic
gctcttccgatctgggttcccaagctggggagtcatttccag

SEQ ID NO: 15 - DNA, 42 bp, Artificial Sequence
  Synthetic
gctcttccgatctgggttcctctatgatgcccactttgtgcc

SEQ ID NO: 16 - DNA, 48 bp, Artificial Sequence
  Synthetic
gctcttccgatctgggttcccaacacagaacttcagcttgatgcagat

SEQ ID NO: 17 - DNA, 45 bp, Artificial Sequence
  Synthetic
gctcttccgatctgggttcctctgtgtcctattctgagccagtcc

SEQ ID NO: 18 - DNA, 45 bp, Artificial Sequence
  Synthetic
gctcttccgatctgggttcccaaactatttcctgagtgcctggca

SEQ ID NO: 19 - DNA, 68 bp, Artificial Sequence
  Synthetic
aatgatacggcgaccaccgagatctacactctttccctacacgacgctcttccgatctgggttcccaa

SEQ ID NO: 20 - DNA, 78 bp, Artificial Sequence
  Synthetic
caagcagaagacggcatacgagatttgacaccgtgtgactggagttcagacgtgtgctcttccgatctgggttcctct

SEQ ID NO: 21 - DNA, 78 bp, Artificial Sequence
  Synthetic
caagcagaagacggcatacgagattcagctgctagtgactggagttcagacgtgtgctcttccgatctgggttcctct

SEQ ID NO: 22 - DNA, 78 bp, Artificial Sequence
  Synthetic
caagcagaagacggcatacgagatcgcgtactatgtgactggagttcagacgtgtgctcttccgatctgggttcctct

SEQ ID NO: 23 - DNA, 78 bp, Artificial Sequence
  Synthetic
caagcagaagacggcatacgagatcgcatcgaaggtgactggagttcagacgtgtgctcttccgatctgggttcctct

SEQ ID NO: 24 - DNA, 78 bp, Artificial Sequence
  Synthetic
caagcagaagacggcatacgagatggttacgacagtgactggagttcagacgtgtgctcttccgatctgggttcctct

SEQ ID NO: 25 - DNA, 78 bp, Artificial Sequence
  Synthetic
caagcagaagacggcatacgagatcaacgtgttagtgactggagttcagacgtgtgctcttccgatctgggttcctct

SEQ ID NO: 26 - DNA, 78 bp, Artificial Sequence
  Synthetic
caagcagaagacggcatacgagatacatatgcgcgtgactggagttcagacgtgtgctcttccgatctgggttcctct

SEQ ID NO: 27 - DNA, 78 bp, Artificial Sequence
  Synthetic
caagcagaagacggcatacgagattacactggtggtgactggagttcagacgtgtgctcttccgatctgggttcctct

SEQ ID NO: 28 - DNA, 78 bp, Artificial Sequence
  Synthetic
caagcagaagacggcatacgagatcatctggctagtgactggagttcagacgtgtgctcttccgatctgggttcctct

SEQ ID NO: 29 - DNA, 78 bp, Artificial Sequence
  Synthetic
caagcagaagacggcatacgagatgtgtcaaccggtgactggagttcagacgtgtgctcttccgatctgggttcctct

SEQ ID NO: 30 - DNA, 10 bp, Artificial Sequence
  Synthetic
acggtgtcaa

SEQ ID NO: 31 - DNA, 10 bp, Artificial Sequence
  Synthetic
tagcagctga

SEQ ID NO: 32 - DNA, 10 bp, Artificial Sequence
  Synthetic
atagtacgcg

SEQ ID NO: 33 - DNA, 10 bp, Artificial Sequence
  Synthetic
cttcgatgcg

SEQ ID NO: 34 - DNA, 10 bp, Artificial Sequence
  Synthetic
tgtcgtaacc

SEQ ID NO: 35 - DNA, 10 bp, Artificial Sequence
  Synthetic
taacacgttg

SEQ ID NO: 36 - DNA, 10 bp, Artificial Sequence
  Synthetic
gcgcatatgt

SEQ ID NO: 37 - DNA, 10 bp, Artificial Sequence
  Synthetic
caccagtgta

SEQ ID NO: 38 - DNA, 10 bp, Artificial Sequence
  Synthetic
tagccagatg

SEQ ID NO: 39 - DNA, 10 bp, Artificial Sequence
  Synthetic
cggttgacac

SEQ ID NO: 40 - DNA, 301 bp, Artificial Sequence
  Synthetic
gtgattcctcgcagaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgaggtgagcggcgtcgcccc

SEQ ID NO: 41 - DNA, 208 bp, Artificial Sequence
  Synthetic
actcccttgccttcttttgattcacatcctaatgccagcaaatacttatgtttttgctatttcagttccatttccataaaatttattttatcatcttttctcataaatttatgccctctatttttactcccaatctgtttaagatgaacaaatcttataaggccacatagctgactgttatttctgttggactccaggaaggagaacc

SEQ ID NO: 42 - DNA, 248 bp, Artificial Sequence
  Synthetic
cacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgtaaggggatattgag

SEQ ID NO: 43 - DNA, 150 bp, Artificial Sequence
  Synthetic
gatgatgcccactttgtgccacatgatggtggctactgcctgtaggcattttccagtgactgaaagaggctgctagtggtagggatgaggtatcatccaatttcctaaaaagattgaacccttcatattcaccagaagagtaacagctgt

SEQ ID NO: 44 - DNA, 214 bp, Artificial Sequence
  Synthetic
ttctgagccagtcctgagaggaaaggaagtataatcaatttgttattaaccaatgaaagaattaagtgaaagataaatctcaggaagcagagggaagtaaacctaatttctgactaagaaagctaaatactatgataacttattcattccttcttttgttcaattacattatttaatcataagtccatgacgtgccaggcactcaggaaatagt

SEQ ID NO: 45 - DNA, 59 bp, Artificial Sequence
  Synthetic
agtggtagggatgaggtatcatccaatttcctaaaaagattgaacccttcatattcacc

SEQ ID NO: 46 - DNA, 59 bp, Artificial Sequence; identical to SEQ ID NO: 45
  Synthetic

SEQ ID NO: 47 - DNA, 98 bp, Artificial Sequence
  Synthetic
gttattaactgatgaagaattaagtgaaagataaccttaggaagcagagggaagttaatctatgactaagaaagttaagtactctgataactcattca

SEQ ID NO: 48 - DNA, 105 bp, Artificial Sequence
  Synthetic
gttattaaccgatgaaagaattaagtgaaagataaatctcaggaagcagagggaagtaaacctaatctctgactaagaaagctaaatactatgataactcattca

SEQ ID NO: 49 - DNA, 105 bp, Artificial Sequence
  Synthetic
gttattaaccaatgaaagaattaagtgaaagataaatctcaggaagcagagggaagtaaacctaatttctgactaagaaagctaaatactatgataacttattca

SEQ ID NO: 50 - DNA, 105 bp, Artificial Sequence; identical to SEQ ID NO: 49
  Synthetic

SEQ ID NO: 51 - DNA, 45 bp, Artificial Sequence
  Synthetic
gctcttccgatctgggttccgtgattccccgcagaggatttcgtg

SEQ ID NO: 52 - DNA, 45 bp, Artificial Sequence
  Synthetic
gctcttccgatctgggttccgtgattcctcgcagaggatttcgtg

SEQ ID NO: 53 - DNA, 39 bp, Artificial Sequence
  Synthetic
gctcttccgatctgggttccgggcgacgccgctcacctc

SEQ ID NO: 54 - DNA, 39 bp, Artificial Sequence
  Synthetic
gctcttccgatctgggttccgggcgacgacgctcacctc

SEQ ID NO: 55 - DNA, 37 bp, Artificial Sequence
  Synthetic
gctcttccgatctgggttcccccttgccttcttttga

SEQ ID NO: 56 - DNA, 37 bp, Artificial Sequence
  Synthetic
gctcttccgatctgggttccggttctccttcctggag

SEQ ID NO: 57 - DNA, 38 bp, Artificial Sequence
  Synthetic
gctcttccgatctgggttcccctgtctgttactgccct

SEQ ID NO: 58 - DNA, 39 bp, Artificial Sequence
  Synthetic
gctcttccgatctgggttccgctggggagtcatttccag

SEQ ID NO: 59 - DNA, 39 bp, Artificial Sequence
  Synthetic
gctcttccgatctgggttccatgatgcccactttgtgcc

SEQ ID NO: 60 - DNA, 45 bp, Artificial Sequence
  Synthetic
gctcttccgatctgggttcccacagaacttcagcttgatgcagat

SEQ ID NO: 61 - DNA, 42 bp, Artificial Sequence
  Synthetic
gctcttccgatctgggttccgtgtcctattctgagccagtcc

SEQ ID NO: 62 - DNA, 42 bp, Artificial Sequence
  Synthetic
gctcttccgatctgggttccactatttcctgagtgcctggca

SEQ ID NO: 63 - DNA, 65 bp, Artificial Sequence
  Synthetic
aatgatacggcgaccaccgagatctacactctttccctacacgacgctcttccgatctgggttcc

SEQ ID NO: 64 - DNA, 75 bp, Artificial Sequence
  Synthetic
caagcagaagacggcatacgagatcgtgagccttgtgactggagttcagacgtgtgctcttccgatctgggttcc

SEQ ID NO: 65 - DNA, 75 bp, Artificial Sequence
  Synthetic
caagcagaagacggcatacgagatgaacgtcagcgtgactggagttcagacgtgtgctcttccgatctgggttcc

SEQ ID NO: 66 - DNA, 75 bp, Artificial Sequence
  Synthetic
caagcagaagacggcatacgagatgtcagattgagtgactggagttcagacgtgtgctcttccgatctgggttcc

SEQ ID NO: 67 - DNA, 75 bp, Artificial Sequence
  Synthetic
caagcagaagacggcatacgagatacgttccagggtgactggagttcagacgtgtgctcttccgatctgggttcc

SEQ ID NO: 68 - DNA, 75 bp, Artificial Sequence
  Synthetic
caagcagaagacggcatacgagatgtaagattgcgtgactggagttcagacgtgtgctcttccgatctgggttcc

SEQ ID NO: 69 - DNA, 46 bp, Artificial Sequence
  Synthetic
gctcttccgatctgggttcctgtgattccccgcagaggatttcgtg

SEQ ID NO: 70 - DNA, 46 bp, Artificial Sequence
  Synthetic
gctcttccgatctgggttcctgtgattcctcgcagaggatttcgtg

SEQ ID NO: 71 - DNA, 40 bp, Artificial Sequence
  Synthetic
gctcttccgatctgggttcccgggcgacgccgctcacctc

SEQ ID NO: 72 - DNA, 40 bp, Artificial Sequence
  Synthetic
gctcttccgatctgggttcccgggcgacgacgctcacctc

SEQ ID NO: 73 - DNA, 38 bp, Artificial Sequence
  Synthetic
gctcttccgatctgggttcctcccttgccttcttttga

SEQ ID NO: 74 - DNA, 38 bp, Artificial Sequence
  Synthetic
gctcttccgatctgggttcccggttctccttcctggag

SEQ ID NO: 75 - DNA, 39 bp, Artificial Sequence
  Synthetic
gctcttccgatctgggttcctcctgtctgttactgccct

SEQ ID NO: 76 - DNA, 40 bp, Artificial Sequence
  Synthetic
gctcttccgatctgggttcccgctggggagtcatttccag

SEQ ID NO: 77 - DNA, 40 bp, Artificial Sequence
  Synthetic
gctcttccgatctgggttcctatgatgcccactttgtgcc

SEQ ID NO: 78 - DNA, 46 bp, Artificial Sequence
  Synthetic
gctcttccgatctgggttccccacagaacttcagcttgatgcagat

SEQ ID NO: 79 - DNA, 43 bp, Artificial Sequence
  Synthetic
gctcttccgatctgggttcctgtgtcctattctgagccagtcc

SEQ ID NO: 80 - DNA, 43 bp, Artificial Sequence
  Synthetic
gctcttccgatctgggttcccactatttcctgagtgcctggca

SEQ ID NO: 81 - DNA, 66 bp, Artificial Sequence
  Synthetic
aatgatacggcgaccaccgagatctacactctttccctacacgacgctcttccgatctgggttccc

SEQ ID NO: 82 - DNA, 76 bp, Artificial Sequence
  Synthetic
caagcagaagacggcatacgagattgtgcacgaagtgactggagttcagacgtgtgctcttccgatctgggttcct

SEQ ID NO: 83 - DNA, 76 bp, Artificial Sequence
  Synthetic
caagcagaagacggcatacgagatccatattagggtgactggagttcagacgtgtgctcttccgatctgggttcct

SEQ ID NO: 84 - DNA, 76 bp, Artificial Sequence
  Synthetic
caagcagaagacggcatacgagatggagactcatgtgactggagttcagacgtgtgctcttccgatctgggttcct

SEQ ID NO: 85 - DNA, 76 bp, Artificial Sequence
  Synthetic
caagcagaagacggcatacgagattccaatgagggtgactggagttcagacgtgtgctcttccgatctgggttcct

SEQ ID NO: 86 - DNA, 76 bp, Artificial Sequence
  Synthetic
caagcagaagacggcatacgagatcactgagagtgtgactggagttcagacgtgtgctcttccgatctgggttcct

SEQ ID NO: 87 - DNA, 47 bp, Artificial Sequence
  Synthetic
gctcttccgatctgggttcctcgtgattccccgcagaggatttcgtg

SEQ ID NO: 88 - DNA, 47 bp, Artificial Sequence
  Synthetic
gctcttccgatctgggttcctcgtgattcctcgcagaggatttcgtg

SEQ ID NO: 89 - DNA, 41 bp, Artificial Sequence
  Synthetic
gctcttccgatctgggttcccagggcgacgccgctcacctc

SEQ ID NO: 90 - DNA, 41 bp, Artificial Sequence
  Synthetic
gctcttccgatctgggttcccagggcgacgacgctcacctc

SEQ ID NO: 91 - DNA, 39 bp, Artificial Sequence
  Synthetic
gctcttccgatctgggttcctccccttgccttcttttga

SEQ ID NO: 92 - DNA, 39 bp, Artificial Sequence
  Synthetic
gctcttccgatctgggttcccaggttctccttcctggag

SEQ ID NO: 93 - DNA, 40 bp, Artificial Sequence
  Synthetic
gctcttccgatctgggttcctccctgtctgttactgccct

SEQ ID NO: 94 - DNA, 41 bp, Artificial Sequence
  Synthetic
gctcttccgatctgggttcccagctggggagtcatttccag

SEQ ID NO: 95 - DNA, 41 bp, Artificial Sequence
  Synthetic
gctcttccgatctgggttcctcatgatgcccactttgtgcc

SEQ ID NO: 96 - DNA, 47 bp, Artificial Sequence
  Synthetic
gctcttccgatctgggttcccacacagaacttcagcttgatgcagat

SEQ ID NO: 97 - DNA, 44 bp, Artificial Sequence
  Synthetic
gctcttccgatctgggttcctcgtgtcctattctgagccagtcc

SEQ ID NO: 98 - DNA, 44 bp, Artificial Sequence
  Synthetic
gctcttccgatctgggttcccaactatttcctgagtgcctggca

SEQ ID NO: 99 - DNA, 67 bp, Artificial Sequence
  Synthetic
aatgatacggcgaccaccgagatctacactctttccctacacgacgctcttccgatctgggttccca

SEQ ID NO: 100 - DNA, 77 bp, Artificial Sequence
  Synthetic
caagcagaagacggcatacgagataggatactctgtgactggagttcagacgtgtgctcttccgatctgggttcctc

SEQ ID NO: 101 - DNA, 75 bp, Artificial Sequence
  Synthetic
caagcagaagacggcatacgagatttagcgtcaagtgactggagttcagacgtgtgctcttccgatctgggttcc

SEQ ID NO: 102 - DNA, 77 bp, Artificial Sequence
  Synthetic
caagcagaagacggcatacgagatatcgttgacagtgactggagttcagacgtgtgctcttccgatctgggttcctc

SEQ ID NO: 103 - DNA, 77 bp, Artificial Sequence
  Synthetic
caagcagaagacggcatacgagatatccagagctgtgactggagttcagacgtgtgctcttccgatctgggttcctc

SEQ ID NO: 104 - DNA, 77 bp, Artificial Sequence
  Synthetic
caagcagaagacggcatacgagatataggccttcgtgactggagttcagacgtgtgctcttccgatctgggttcctc

SEQ ID NO: 105 - DNA, 48 bp, Artificial Sequence; identical to SEQ ID NO: 7
  Synthetic

SEQ ID NO: 106 - DNA, 48 bp, Artificial Sequence; identical to SEQ ID NO: 8
  Synthetic

SEQ ID NO: 107 - DNA, 42 bp, Artificial Sequence; identical to SEQ ID NO: 9
  Synthetic

SEQ ID NO: 108 - DNA, 42 bp, Artificial Sequence; identical to SEQ ID NO: 10
  Synthetic

SEQ ID NO: 109 - DNA, 40 bp, Artificial Sequence; identical to SEQ ID NO: 11
  Synthetic

SEQ ID NO: 110 - DNA, 40 bp, Artificial Sequence; identical to SEQ ID NO: 12
  Synthetic

SEQ ID NO: 111 - DNA, 41 bp, Artificial Sequence; identical to SEQ ID NO: 13
  Synthetic

SEQ ID NO: 112 - DNA, 42 bp, Artificial Sequence; identical to SEQ ID NO: 14
  Synthetic

SEQ ID NO: 113 - DNA, 42 bp, Artificial Sequence; identical to SEQ ID NO: 15
  Synthetic

SEQ ID NO: 114 - DNA, 48 bp, Artificial Sequence; identical to SEQ ID NO: 16
  Synthetic

SEQ ID NO: 115 - DNA, 45 bp, Artificial Sequence; identical to SEQ ID NO: 17
  Synthetic

SEQ ID NO: 116 - DNA, 45 bp, Artificial Sequence; identical to SEQ ID NO: 18
  Synthetic

SEQ ID NO: 117 - DNA, 78 bp, Artificial Sequence; identical to SEQ ID NO: 20
  Synthetic

SEQ ID NO: 118 - DNA, 68 bp, Artificial Sequence; identical to SEQ ID NO: 19
  Synthetic

SEQ ID NO: 119 - DNA, 78 bp, Artificial Sequence; identical to SEQ ID NO: 20
  Synthetic

SEQ ID NO: 120 - DNA, 78 bp, Artificial Sequence; identical to SEQ ID NO: 21
  Synthetic

SEQ ID NO: 121 - DNA, 78 bp, Artificial Sequence; identical to SEQ ID NO: 22
  Synthetic

SEQ ID NO: 122 - DNA, 78 bp, Artificial Sequence; identical to SEQ ID NO: 23
  Synthetic

SEQ ID NO: 123 - DNA, 78 bp, Artificial Sequence; identical to SEQ ID NO: 24
  Synthetic

SEQ ID NO: 124 - DNA, 51 bp, Artificial Sequence
  Synthetic
gctcttccgatctgggttcctctatcgtgattccccgcagaggatttcgtg

SEQ ID NO: 125 - DNA, 51 bp, Artificial Sequence
  Synthetic
gctcttccgatctgggttcctctatcgtgattcctcgcagaggatttcgtg

SEQ ID NO: 126 - DNA, 45 bp, Artificial Sequence
  Synthetic
gctcttccgatctgggttcccaactagggcgacgccgctcacctc

SEQ ID NO: 127 - DNA, 45 bp, Artificial Sequence
  Synthetic
gctcttccgatctgggttcccaactagggcgacgacgctcacctc

SEQ ID NO: 128 - DNA, 43 bp, Artificial Sequence
  Synthetic
gctcttccgatctgggttcctctatccccttgccttcttttga

SEQ ID NO: 129 - DNA, 43 bp, Artificial Sequence
  Synthetic
gctcttccgatctgggttcccaactaggttctccttcctggag

SEQ ID NO: 130 - DNA, 44 bp, Artificial Sequence
  Synthetic
gctcttccgatctgggttcctctatccctgtctgttactgccct

SEQ ID NO: 131 - DNA, 45 bp, Artificial Sequence
  Synthetic
gctcttccgatctgggttcccaactagctggggagtcatttccag

SEQ ID NO: 132 - DNA, 45 bp, Artificial Sequence
  Synthetic
gctcttccgatctgggttcctctatcatgatgcccactttgtgcc

SEQ ID NO: 133 - DNA, 51 bp, Artificial Sequence
  Synthetic
gctcttccgatctgggttcccaactacacagaacttcagcttgatgcagat

SEQ ID NO: 134 - DNA, 48 bp, Artificial Sequence
  Synthetic
gctcttccgatctgggttcctctatcgtgtcctattctgagccagtcc

SEQ ID NO: 135 - DNA, 48 bp, Artificial Sequence
  Synthetic
gctcttccgatctgggttcccaactaactatttcctgagtgcctggca

SEQ ID NO: 136 - DNA, 71 bp, Artificial Sequence
  Synthetic
aatgatacggcgaccaccgagatctacactctttccctacacgacgctcttccgatctgggttcccaacta

SEQ ID NO: 137 - DNA, 81 bp, Artificial Sequence
  Synthetic
caagcagaagacggcatacgagatgcgtaacatcgtgactggagttcagacgtgtgctcttccgatctgggttcctctatc

SEQ ID NO: 138 - DNA, 81 bp, Artificial Sequence
  Synthetic
caagcagaagacggcatacgagatgtagtgcatagtgactggagttcagacgtgtgctcttccgatctgggttcctctatc

SEQ ID NO: 139 - DNA, 81 bp, Artificial Sequence
  Synthetic
caagcagaagacggcatacgagatttaaggactggtgactggagttcagacgtgtgctcttccgatctgggttcctctatc

SEQ ID NO: 140 - DNA, 81 bp, Artificial Sequence
  Synthetic
caagcagaagacggcatacgagatgatctagcgagtgactggagttcagacgtgtgctcttccgatctgggttcctctatc

SEQ ID NO: 141 - DNA, 81 bp, Artificial Sequence
  Synthetic
caagcagaagacggcatacgagatctgaccgaatgtgactggagttcagacgtgtgctcttccgatctgggttcctctatc

SEQ ID NO: 142 - DNA, 60 bp, Artificial Sequence
  Synthetic
gctcttccgatctgggttcctctatctcctaacctgtgattccccgcagaggatttcgtg

SEQ ID NO: 143 - DNA, 60 bp, Artificial Sequence
  Synthetic
gctcttccgatctgggttcctctatctcctaacctgtgattcctcgcagaggatttcgtg

SEQ ID NO: 144 - DNA, 54 bp, Artificial Sequence
  Synthetic
gctcttccgatctgggttcccaactaatcaaccaagggcgacgccgctcacctc

SEQ ID NO: 145 - DNA, 54 bp, Artificial Sequence
  Synthetic
gctcttccgatctgggttcccaactaatcaaccaagggcgacgacgctcacctc

SEQ ID NO: 146 - DNA, 52 bp, Artificial Sequence
  Synthetic
gctcttccgatctgggttcctctatctcctaacctcccttgccttcttttga

SEQ ID NO: 147 - DNA, 52 bp, Artificial Sequence
  Synthetic
gctcttccgatctgggttcccaactaatcaaccaaggttctccttcctggag

SEQ ID NO: 148 - DNA, 53 bp, Artificial Sequence
  Synthetic
gctcttccgatctgggttcctctatctcctaacctcctgtctgttactgccct

SEQ ID NO: 149 - DNA, 54 bp, Artificial Sequence
  Synthetic
gctcttccgatctgggttcccaactaatcaaccaagctggggagtcatttccag

SEQ ID NO: 150 - DNA, 54 bp, Artificial Sequence
  Synthetic
gctcttccgatctgggttcctctatctcctaacctatgatgcccactttgtgcc

SEQ ID NO: 151 - DNA, 60 bp, Artificial Sequence
  Synthetic
gctcttccgatctgggttcccaactaatcaaccaacacagaacttcagcttgatgcagat

SEQ ID NO: 152 - DNA, 57 bp, Artificial Sequence
  Synthetic
gctcttccgatctgggttcctctatctcctaacctgtgtcctattctgagccagtcc

SEQ ID NO: 153 - DNA, 57 bp, Artificial Sequence
  Synthetic
gctcttccgatctgggttcccaactaatcaaccaaactatttcctgagtgcctggca

SEQ ID NO: 154 - DNA, 80 bp, Artificial Sequence
  Synthetic
aatgatacggcgaccaccgagatctacactctttccctacacgacgctcttccgatctgggttcccaactaatcaaccaa

SEQ ID NO: 155 - DNA, 90 bp, Artificial Sequence
  Synthetic
caagcagaagacggcatacgagatacgactcgatgtgactggagttcagacgtgtgctcttccgatctgggttcctctatctcctaacct

SEQ ID NO: 156 - DNA, 90 bp, Artificial Sequence
  Synthetic
caagcagaagacggcatacgagatctcaatcggagtgactggagttcagacgtgtgctcttccgatctgggttcctctatctcctaacct

SEQ ID NO: 157 - DNA, 90 bp, Artificial Sequence
  Synthetic
caagcagaagacggcatacgagatacaattcgtggtgactggagttcagacgtgtgctcttccgatctgggttcctctatctcctaacct

SEQ ID NO: 158 - DNA, 90 bp, Artificial Sequence
  Synthetic
caagcagaagacggcatacgagattacttgcgacgtgactggagttcagacgtgtgctcttccgatctgggttcctctatctcctaacct

SEQ ID NO: 159 - DNA, 90 bp, Artificial Sequence
  Synthetic
caagcagaagacggcatacgagatacttaaccgtgtgactggagttcagacgtgtgctcttccgatctgggttcctctatctcctaacct

(19) United States
(12) Patent Application Publication
(10) Pub. No.: US 2020/0291453 A1
(43) Pub. Date: Sep. 17, 2020

(54) AMPLICON GENERATION

(71) Applicant: Mayo Foundation for Medical Education and Research, Rochester, MN (US)

(72) Inventors: Brian A. Dukek, Rochester, MN (US); Manish J. Gandhi, Rochester, MN (US)

(21) Appl. No.: 16/743,377

(22) Filed: Jan. 15, 2020

Related U.S. Application Data
(60) Provisional application No. 62/798,163, filed on Jan. 29, 2019.

(51) Int. Cl. C12Q 1/686

(52) U.S. Cl. CPC C12Q 1/686

(57) ABSTRACT

The invention provides compositions and methods for accurately and specifically amplifying sequences to allow for accurate and specific detection of mutations, such as in disease-associated genes and alleles, thus distinguishing true nucleotide variants over random nucleotide sequencing errors. In one embodiment, this is accomplished, prior to sequencing, by using a combination of director and driver in a combination of two PCR reactions. Thus, in one embodiment, this is accomplished by amplifying a nucleotide sequence of interest to introduce a director that creates a specific target for a subsequent amplification in which a driver that specifically hybridizes to the director drives the specificity of further amplification. The amplification produces an amplicon of a sense or antisense strand of a double-stranded nucleotide sequence of interest. The amplicon may optionally contain universal sequences and/or index sequences that facilitate subsequent sequencing of the amplicon, such as using sequencing-by-synthesis. The reagents of the first and second amplification steps may be combined in a single reaction mixture.

### CROSS-REFERENCE TO RELATED APPLICATIONS

This application claims priority under 35 U.S.C. § 119(e) to co-pending U.S. Provisional Patent Application Ser. No. 62/798,163, filed Jan. 29, 2019, incorporated by reference.

### SEQUENCE LISTING

A Sequence Listing has been submitted in an ASCII text file named “19594” created on May 10, 2020, consisting of 34,802 bytes, the entire content of which is herein incorporated by reference.

### FIELD OF THE INVENTION

The invention provides compositions and methods for accurately and specifically amplifying sequences to allow for accurate and specific detection of mutations, such as in disease-associated genes and alleles, thus distinguishing true nucleotide variants over random nucleotide sequencing errors. In one embodiment, this is accomplished, prior to sequencing, by using a combination of director and driver in a combination of two PCR reactions. Thus, in one embodiment, this is accomplished by first amplifying a nucleotide sequence of interest to introduce a director that creates a specific target for a second amplification in which a driver, which specifically hybridizes to the director, drives the directionality of the second amplification. The amplification produces an amplicon of a sense or antisense strand of a double-stranded nucleotide sequence of interest. The amplicon may optionally contain universal sequences and/or index sequences that facilitate subsequent sequencing of the amplicon, such as using sequencing-by-synthesis. The reagents of the first and second amplification steps may be combined in a single reaction mixture.

### BACKGROUND OF THE INVENTION

Genetic testing of disease relies on accurately and specifically detecting disease-associated mutations and/or disease-associated alleles, particularly hypervariable alleles, such as those in the HLA DQA and HLA DQB genes associated with celiac disease. Testing for celiac disease is one of the highest volume assays, costly, and time consuming, requiring a PCR, a gel, hybridization and washing of beads, and reading on instruments. Thus, even small cost savings in this assay would lead to large overall savings.

Current genetic testing for celiac disease can rule out celiac disease, can indicate an individual is at risk to develop celiac disease, but cannot, alone, directly diagnose celiac disease.

Thus, there remains a need for compositions and methods for accurately and specifically amplifying sequences so that disease-associated mutations and/or disease-associated alleles may be accurately and specifically sequences for disease detection.

### SUMMARY OF THE INVENTION

The invention provides compositions and methods for accurately and specifically amplifying sequences to allow for accurate and specific detection of mutations, such as in disease-associated genes and alleles, thus distinguishing true nucleotide variants over random nucleotide sequencing errors. In one embodiment, this is accomplished, prior to sequencing, by using a combination of director and driver in a combination of two PCR reactions. Thus, in one embodiment, this is accomplished by first amplifying a nucleotide sequence of interest to introduce a director that creates a specific target for a second amplification in which a driver, which specifically hybridizes to the director's complementary strand product, drives the directionality of the second amplification. The amplification produces an amplicon of a sense or antisense strand of a double-stranded nucleotide sequence of interest. The amplicon may optionally contain universal sequences and/or index sequences that facilitate subsequent sequencing of the amplicon, such as using sequencing-by-synthesis. The reagents of the first and second amplification steps may be combined in a single reaction mixture.

Thus, in one embodiment, the invention provides a method for amplifying a target polynucleotide sequence, comprising A) contacting a double-stranded target polynucleotide sequence with i) a first primer modified by having an insertion of a first director, and ii) a second primer, said contacting is under conditions sufficient for amplifying said double-stranded target polynucleotide sequence to produce a first plurality of amplicons comprising a first single-stranded amplicon that comprises at least one single strand of said double-stranded target polynucleotide sequence, said at least one single strand having an insertion of said first director in either its 3′ or 5′ terminal regions, and B) contacting said at least one single strand with i) a third primer fused at its 3′ end to a first driver, and ii) a fourth primer, wherein said first driver has the same sequence as said first director, wherein said contacting is under conditions sufficient for amplifying said at least one single strand produced in step A) to produce a second plurality of amplicons comprising a second single-stranded amplicon having at its 3′ end at least one single strand containing an insertion of said first director in either its 3′ or 5′ terminal regions. In one embodiment, the 5′ end of said first driver is fused to a universal adapter, and wherein said second single-stranded amplicon comprises said universal adapter sequence fused to its 5′ end.

Thus, in another embodiment, the invention provides a method for amplifying a target polynucleotide sequence, comprising A) contacting a double-stranded target polynucleotide sequence with i) a first primer modified by having an insertion of a first director, and ii) a second primer modified by having an insertion of a second director, said contacting is under conditions sufficient for amplifying said double-stranded target polynucleotide sequence to produce a first plurality of amplicons comprising a first single-stranded amplicon that comprises at least one single strand of said double-stranded target polynucleotide sequence, said at least one single strand having an insertion of said first director in either its 3′ or 5′ terminal regions and an insertion of said complement of said second director in its other terminal region, and B) contacting said at least one single strand with i) a third primer fused at its 3′ end to a first driver, and ii) a fourth primer fused at its 3′ end to a second driver, wherein said first driver has the same sequence as said first director, and wherein said second driver has the same sequence as said second director, and wherein said contacting is under conditions sufficient for amplifying said at least one single strand produced in step A) to produce a second plurality of amplicons comprising a second single-stranded amplicon having at its 3′ end said at least one single strand containing an insertion of said first director in either its 3′ or 5′ terminal regions, and containing an insertion of said second director in its other terminal region. In one embodiment, the 5′ end of said first driver is fused to a universal adapter, or the 5′ end of said second driver is fused to a complement of said universal adapter sequence, and wherein said second single-stranded amplicon comprises said universal adapter sequence fused to its 5′ end. In one embodiment, said contacting of steps A) and B) is in a single reaction mixture. In one embodiment, said first primer and said third primer are forward primers, and said second primer and said fourth primer are reverse primers. In one embodiment, said first primer and said third primer are reverse primers, and said second primer and said fourth primer are forward primers. In one embodiment, said second single-stranded amplicon contains fused in operable combination from the 5′ end to the 3′ end, a) said universal adapter sequence, b) an insertion of said first director in either its 3′ or 5′ terminal regions, c) said nucleotide sequence of interest, d) an insertion of said second director in its other terminal region, and e) an optional unique index sequence. In another embodiment, said second single-stranded amplicon contains fused in operable combination from the 5′ end to the 3′ end, a) an optional unique index sequence, b) an insertion of said first director in either its 3′ or 5′ terminal regions, c) said nucleotide sequence of interest, d) an insertion of said second director in its other terminal region, and e) said universal adapter sequence. In one embodiment, the 5′ end of said first driver of said third primer is fused to the complement of a unique index sequence and the 5′ end of said second driver of said fourth primer is fused to said universal adapter sequence. In one embodiment, said second single-stranded amplicon is fused at its 3′ end to said unique index sequence, and fused at its 5′ end to said universal adapter sequence. In one embodiment, the 5′ end of said first driver of said third primer is fused to the complement of said universal adapter sequence and the 5′ end of said second driver of said fourth primer is fused to a unique index sequence. In one embodiment, said second single-stranded amplicon is fused at its 5′ end to said unique index sequence, and fused at its 3′ end to said universal adapter sequence. In one embodiment, said second single-stranded amplicon contains an insertion in its 5′ terminal region of a sense strand of said unique index sequence, and an insertion in its 3′ terminal region of a sense strand of said universal adapter sequence. In one embodiment, the method further comprises sequencing said single-stranded amplicon comprised in said second plurality of amplicons. In one embodiment, said conditions in steps A) and B) are sufficient to produce said second single-stranded amplicon at a higher efficiency than in the absence of one or more of said director, said first driver, and said second driver. In one embodiment, said conditions in steps A) and/or B) comprise denaturing said double-stranded target polynucleotide sequence into a single-stranded sense sequence and single-stranded antisense sequence. The contacting step need not be with a double-stranded target polynucleotide sequence, but may be with a single-stranded target derived from a double-stranded target polynucleotide sequence. In one embodiment, said conditions sufficient for amplifying said double-stranded target polynucleotide sequence to produce a first plurality of amplicons comprise one or more of polymerase chain reaction (PCR) amplification, isothermal amplifications, denaturing said double-stranded target polynucleotide sequence, annealing one or more of said first and second primers to said double-stranded target polynucleotide sequence, and extending the annealed primer. In one embodiment, said conditions sufficient for amplifying said at least one single strand produced in step A) to produce a second plurality of amplicons comprise one or more of polymerase chain reaction (PCR) amplification, isothermal amplifications, denaturing said double-stranded target polynucleotide sequence, annealing one or more of said third and fourth primers to said at least one single strand produced in step A), and extending the annealed primer. In one embodiment, the concentration of one or both of said first primer and said first primer is 25% or less than the concentration of one or both of said second primer and said second primer. In one embodiment, said amplifying of one or both of steps A) and B) comprises 50 or fewer PCR cycles. In one embodiment, said target polynucleotide sequence comprises genomic DNA. In one embodiment, said genomic DNA comprises a variable sequence of an allele. In one embodiment, said second single-stranded amplicon is an amplified sense strand of said double-stranded target polynucleotide sequence or an amplified antisense strand of said double-stranded target polynucleotide sequence.

The invention also provides a method for amplifying a target polynucleotide sequence, comprising contacting i) a sample comprising a plurality of double-stranded target polynucleotide sequences comprising a first single-stranded polynucleotide sequence and a second single-stranded polynucleotide sequence, and comprising a first portion and a second portion, ii) first primer comprising a first sequence that is complementary to said first portion of said first single-stranded polynucleotide sequence, said first sequence is modified by having an insertion of a director, wherein said director is not complementary either to said first portion of said first single-stranded polynucleotide sequence or to said second portion of said second single-stranded polynucleotide sequence, iii) second primer comprising a second sequence that is complementary to said second portion of said second single-stranded polynucleotide sequence, said second sequence is modified by having an insertion of a second director, wherein neither the first nor second said directors is complementary either to said first portion of said first single-stranded polynucleotide sequence or to said second portion of said second single-stranded polynucleotide sequence, iv) third primer fused at its 3′ end to a first driver, and v) fourth primer fused at its 3′ end to a second driver, wherein said first driver has the same sequence as said first director and said second driver has the same sequence as said second director, and wherein said contacting is under conditions sufficient for hybridizing said first driver with said complement of said first director and second driver with said complement of said second director, and for amplifying said plurality of target polynucleotide sequences to produce a) a first plurality of amplicons comprising a first single-stranded amplicon that comprises i) said first single-stranded polynucleotide sequence having an insertion of said director in either its 3′ or 5′ terminal regions and an insertion of said second director in its other terminal region, or ii) said second single-stranded polynucleotide sequence having an insertion of said director in either its 3′ or 5′ terminal regions and an insertion of said second director in its other terminal region, and b) a second plurality of amplicons comprising a second single-stranded amplicon having at its 3′ end either i) said first single-stranded polynucleotide sequence containing an insertion of said director in either its 3′ or 5′ terminal regions, and containing an insertion of said second director in its other terminal region, or ii) said second single-stranded polynucleotide sequence containing an insertion of said director in either its 3′ or 5′ terminal regions, and containing an insertion of said second director in its other terminal region. In one embodiment, the 5′ end of either said first driver or said second driver is fused to a universal adapter sequence. In one embodiment, said second single-stranded amplicon is an amplified sense strand of said double-stranded target polynucleotide sequence or an amplified antisense strand of said double-stranded target polynucleotide sequence. In one embodiment, said conditions comprise denaturing said double-stranded target polynucleotide sequence into a single-stranded sense sequence and single-stranded antisense sequence. The contacting step need not be with a double-stranded target polynucleotide sequence, but may be with a single-stranded target derived from a double-stranded target polynucleotide sequence. In one embodiment, said conditions sufficient for hybridizing said first driver with the complement of said first director and said second driver with the complement of said second director comprise using a lower temperature than a temperature used to denature said double-stranded target polynucleotide sequence. In one embodiment, said conditions sufficient for amplifying said plurality of target polynucleotide sequence comprise one or more of polymerase chain reaction (PCR) amplification, isothermal amplifications, denaturing said double-stranded target polynucleotide sequence, annealing one or more of said first, second, third and fourth primers to said double-stranded target polynucleotide sequence, and extending the annealed primer. In one embodiment, said contacting of steps c) and e) is in a single reaction mixture. In one embodiment, said first primer and said third primer are forward primers, and said second primer and said fourth primer are reverse primers. In one embodiment, said first primer and said third primer are reverse primers, and said second primer and said fourth primer are forward primers. In one embodiment, said second single-stranded amplicon contains, fused in operable combination from the 5′ end to the 3′ end, universal adapter sequence, and said first single-stranded polynucleotide sequence having an insertion of said director in either its 3′ or 5′ terminal regions and an insertion of said complement of said director in its other terminal region, or said second single-stranded polynucleotide sequence having an insertion of said director in either its 3′ or 5′ terminal regions and an insertion of said complement of said director in its other terminal region. In one embodiment, wherein the 5′ end of said first driver of said third primer is fused to a unique index sequence, and the 5′ end of said second driver of said fourth primer is fused to universal adapter sequence. In one embodiment, said second single-stranded amplicon is fused at its 3′ end to said unique index sequence, and fused at its 5′ end to universal adapter sequence. In one embodiment, said second single-stranded amplicon contains an insertion in its 3′ terminal region of a sense strand of said unique index sequence, and an insertion in its 5′ terminal region of a sense strand of universal adapter sequence. In one embodiment, said second single-stranded amplicon contains, fused in operable combination from the 5′ end to the 3′ end, i) universal adapter sequence, ii) said first single-stranded polynucleotide sequence having an insertion of said director in either its 3′ or 5′ terminal regions and an insertion of said complement of said director in its other terminal region, or said second single-stranded polynucleotide sequence having an insertion of said director in either its 3′ or 5′ terminal regions and an insertion of said complement of said director in its other terminal region, and iii) said unique index sequence. In one embodiment, said unique index sequence is comprised in an index adapter. In one embodiment, the 5′ end of said first driver of said third primer is fused to universal adapter sequence, and the 5′ end of said second driver of said fourth primer is fused to a unique index sequence. In one embodiment, said second single-stranded amplicon is fused at its 5′ end to said unique index sequence, and fused at its 3′ end to universal adapter sequence. In one embodiment, said second single-stranded amplicon contains an insertion in its 5′ terminal region of a sense strand of said unique index sequence, and an insertion in its 3′ terminal region of a sense strand of universal adapter sequence. In one embodiment, said unique index sequence is comprised in an index adapter.

The invention additionally provides a method for amplifying a sense strand of a target polynucleotide sequence, comprising, contacting i) a sample comprising a plurality of target polynucleotide sequences comprising sense and antisense strands, ii) first forward primer comprising a first sequence that is complementary to the sense strand of a first portion of said target polynucleotide sequences, said first sequence is modified by having an insertion of a director, iii) first reverse primer comprising a second sequence that is complementary to the antisense strand of a second portion of said target polynucleotide sequences, said second sequence is modified by having an insertion of a second director, wherein neither first nor second director is complementary either to said first portion or to said second portion of said target polynucleotide sequences, iv) second forward primer fused at its 3′ end to a first driver, v) second reverse primer fused at its 3′ end to a second driver, wherein one of said first driver and said second driver has the same sequence as said director, and the other of said first driver and said second driver has the same sequence as the second director, and wherein said contacting is under conditions sufficient for a) hybridizing said plurality of target polynucleotide sequences with the complement of said first forward primer and said first reverse primer, b) amplifying said plurality of target polynucleotide sequences to produce a first plurality of amplicons comprising a first sense strand amplicon comprising said target polynucleotide sequences having an insertion of said forward strand director in its 5′ terminal region and having an insertion of complement to said reverse strand director in its 3′ terminal region, c) contacting said second forward primer with said first sense strand amplicon, d) hybridizing said reverse strand driver with the complement of said reverse strand director of said first sense strand amplicon, e) contacting said second reverse primer with said first antisense strand amplicon, f) hybridizing said forward strand driver with said complement to said forward strand director of said first antisense strand amplicon, and g) amplifying said first sense strand amplicon and said first antisense strand amplicon to produce a second plurality of amplicons comprising a single-stranded amplicon that contains, fused in operable combination from the 5′ end to the 3′ end, said sense strand universal adapter sequence, and said target polynucleotide sequence having an insertion of said forward strand director in its 5′ terminal region and having an insertion of said complement to said reverse strand director in its 3′ terminal region. In one embodiment, the 5′ end of either said first driver or said second driver is fused to a universal adapter sequence. In one embodiment, said contacting of steps c) and e) is in a single reaction mixture. In one embodiment, said second forward primer comprises a complement to a unique index sequence fused at its 3′ end to said driver, and wherein said single-stranded amplicon contains at its 3′ end a sense strand of said unique index sequence.

The invention also provides a reaction mixture for amplifying a double-stranded target polynucleotide sequence that contains a first single-stranded polynucleotide sequence and a second single-stranded polynucleotide sequence, and contains a first portion and a second portion, said reaction mixture comprising a) first primer comprising a first sequence that is complementary to said first portion of said first single-stranded polynucleotide sequence, said first sequence is modified by having an insertion of a director, b) second primer comprising a second sequence that is complementary to said second portion of said second single-stranded polynucleotide sequence, said second sequence is modified by having an insertion of a complement of a second director, wherein neither said first director or said second director is complementary either to said first portion of said first single-stranded polynucleotide sequence or to said second portion of said second single-stranded polynucleotide sequence, c) third primer fused at its 3′ end to a first driver, and d) fourth primer fused at its 3′ end to a second driver, wherein the first driver has the same sequence as said the first director, and the second driver is a the same sequence as the second director. In one embodiment, said reaction mixture comprises said first sequence hybridized along a portion of its length to said first portion of said first single-stranded polynucleotide sequence, and said second sequence hybridized along a portion of its length to said second portion of said second single-stranded polynucleotide sequence. In one embodiment, said reaction mixture comprises said first driver hybridized to the complement of said first director, and said second driver hybridized to said complement of said second director. In one embodiment, said reaction mixture comprises one or both of the 5′ end of said first driver of said third primer is fused to a unique index sequence, and the 5′ end of said second driver of said fourth primer is fused to said universal adapter sequence. In one embodiment, the 5′ end of said first driver of said third primer is fused to a unique index sequence. In one embodiment, the 5′ end of said second driver of said fourth primer is fused to said universal adapter sequence. In one embodiment, the 5′ end of said first driver of said third primer is fused to said universal adapter sequence. In one embodiment, the 5′ end of said second driver of said fourth primer is fused to a unique index sequence.

The invention further provides a reaction mixture for amplifying a double-stranded target polynucleotide sequence that contains a first single-stranded polynucleotide sequence and a second single-stranded polynucleotide sequence, and contains a first portion and a second portion, said reaction mixture comprising a) first primer comprising a first sequence that is complementary to said first portion of said first single-stranded polynucleotide sequence, said first sequence is modified by having an insertion of a director, and said first sequence hybridized along a portion of its length to said first portion of said first single-stranded polynucleotide sequence, b) second primer comprising a second sequence that is complementary to said second portion of said second single-stranded polynucleotide sequence, said second sequence is modified by having an insertion of a second director, wherein neither said first director or said second director is complementary either to said first portion of said first single-stranded polynucleotide sequence or to said second portion of said second single-stranded polynucleotide sequence, and said second sequence hybridized along a portion of its length to said second portion of said second single-stranded polynucleotide sequence, c) third primer fused at its 3′ end to a first driver, and d) fourth primer fused at its 3′ end to a second driver, wherein said first driver has the same sequence as said first director, and the said second driver has the same sequence as said second director.

Also provided herein is a reaction mixture for amplifying a double-stranded target polynucleotide sequence that contains a first single-stranded polynucleotide sequence and a second single-stranded polynucleotide sequence, and contains a first portion and a second portion, said reaction mixture comprising a) first primer comprising a first sequence that is complementary to said first portion of said first single-stranded polynucleotide sequence, said first sequence is modified by having an insertion of a director, b) second primer comprising a second sequence that is complementary to said second portion of said second single-stranded polynucleotide sequence, said second sequence is modified by having an insertion of a second director, wherein neither said first director nor said second director is complementary either to said first portion of said first single-stranded polynucleotide sequence or to said second portion of said second single-stranded polynucleotide sequence, c) third primer fused at its 3′ end to a first driver, and d) fourth primer fused at its 3′ end to a second driver, wherein said first driver has the same sequence as said first director, and the said second driver has the same sequence as said second director, and wherein said first driver is hybridized to the complement of said first director, and said second driver is hybridized to said complement of said second director.

The invention also provides a kit for a amplifying double-stranded target polynucleotide sequence, said kit comprising any one or more of the reaction mixtures described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of an exemplary embodiment of the invention's methods for production of the sense strand of a target polynucleotide sequence.

FIG. 2 is a schematic of the amplicon produced by the method of FIG. 1 in which the exemplary sense strand of the target polynucleotide sequence is amplified into a sequenceable amplicon. The sequenceable sense strand (i.e., top strand) reads from the 5′ to 3′ end as follows: universal adapter, driver, target polynucleotide sequence, driver, and index adapter containing an exemplary 6-base index region.

FIG. 3 A-B is a schematic of (FIG. 3A) traditional Illumina method, and (FIG. 3B) the invention's methods as exemplified by production of the sense strand of a target polynucleotide sequence

FIG. 4 is a schematic of an initial strategy targeting regions of the hypervariable regions that define specific alleles in the celiac disease HLA DQA and HLA DQB genes.

FIG. 5 shows exemplary PCR primers with associated directors for HLA-DQA1 and HLA-DQB1 amplification, exemplary universal adapter with associated driver, and 10 exemplary antisense indexes with associated drivers used in the invention's methods. As shown in the key, the gene specific region is in light blue, forward 3 bp driver in light green, forward 3 bp director in orange, reverse 3 bp driver in light purple, non-directional PCR primer/adapter interface in red, and index in green.

FIG. 6 shows the exemplary index sequences of FIG. 5 as read by the MiSeq kit cartridge (Illumina, San Diego, Calif.).

FIG. 7 A-B shows region of (FIG. 7A) genomic HLA-DQB1 and (FIG. 7B) genomic HLA-DQA1 genes amplified by PCR, including initial PCR.

FIG. 8 A-B shows sequencing data for (FIG. 8A) a patient with DQB1:03/DQB1:06 (FIG. 8A) and (FIG. 8B) a patient with DQA1:02/DQA1:01:03.

FIG. 9 shows exemplary sequences used in the invention's methods. Black characters are gene target specific, red characters are specific to the Illumina universal sequences, blue characters are index sequences, green highlighting are forward directors, pink highlighting are reverse directors, yellow highlighting are forward drivers, and gray highlighting are reverse drivers.

FIG. 10A-B shows (FIG. 10A) a scatter plot of percent reads associated with each driver length, and (FIG. 10B) a scatter plot of percent reads associated with each driver length, showing the percent of reads on the flowcell (y axis) as affected by the length in base pairs of the driver (x axis) using six driver lengths (0, 1, 2, 3, 6, and 15 bp) on five different patients (S1-S5).

### DEFINITIONS

To facilitate understanding of the invention, a number of terms are defined below.

The term “in a single reaction mixture” when in reference to contacting reagents (such as primers, nucleotide bases, target polynucleotide sequence template, enzymes) of two or more reactions (such as a first reaction of site specific PCR amplification using site specific primers, and a second reaction of PCR amplification using index primers and universal primers) means combining the reagents of the two or more reactions without temporally waiting for, and/or without providing, conditions (e.g., thermal cycling for PCR amplification, such as temperature for denaturing double-stranded nucleotide sequences into to single-stranded sequences, temperature for hybridization of sequences, etc.) that are sufficient for any of the two or more reactions to begin and/or to be completed. The term contacting “in a single reaction mixture” includes sequentially and/or substantially simultaneously adding the reagents to a single vessel or receptacle that allows physical contact between the reagents.

Plurality” refers to a population of two or more different polynucleotides or other referenced molecule. Accordingly, unless expressly stated otherwise, the term “plurality” is used synonymously with “population.” A plurality includes 2, 3, 4, 5, 6, 7, 8, 9, 10, 20, 30, 40, 50, 60, 70, 80, 90 or a 100 or more different members of the population. A plurality also can include 200, 300, 400, 500, 1000, 5000, 10000, 50000, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶ or 1×107, or more different members. A plurality includes all integer numbers in between the above exemplary population numbers.

“Target polynucleotide” means a polynucleotide of interest that is the object of an analysis or action. “Target polynucleotide” includes members of a plurality of target polynucleotide sequences having the same sequence. The analysis or action includes subjecting the polynucleotide to copying, amplification, sequencing and/or other procedure for nucleic acid interrogation. A target polynucleotide is exemplified by a portion of a gene, director, driver sequences, adapter sequences, and/or index sequences.

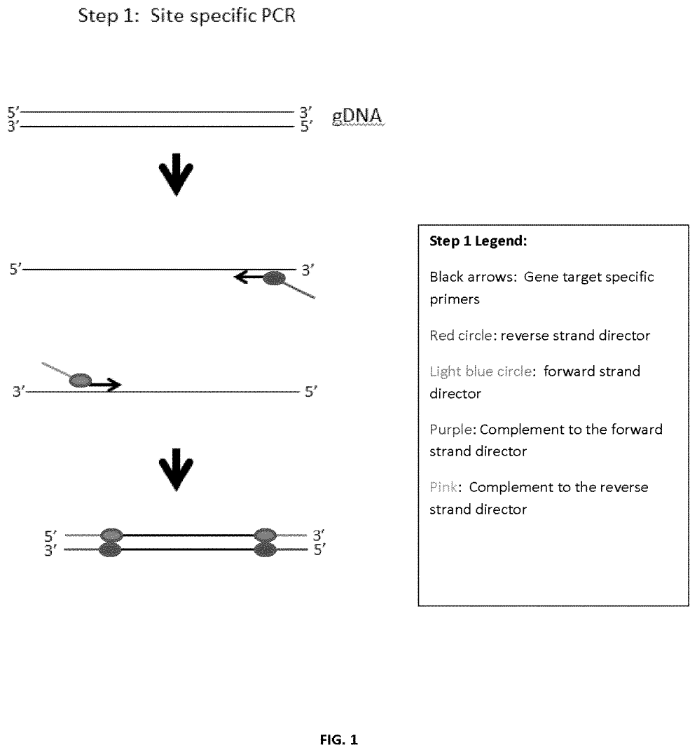

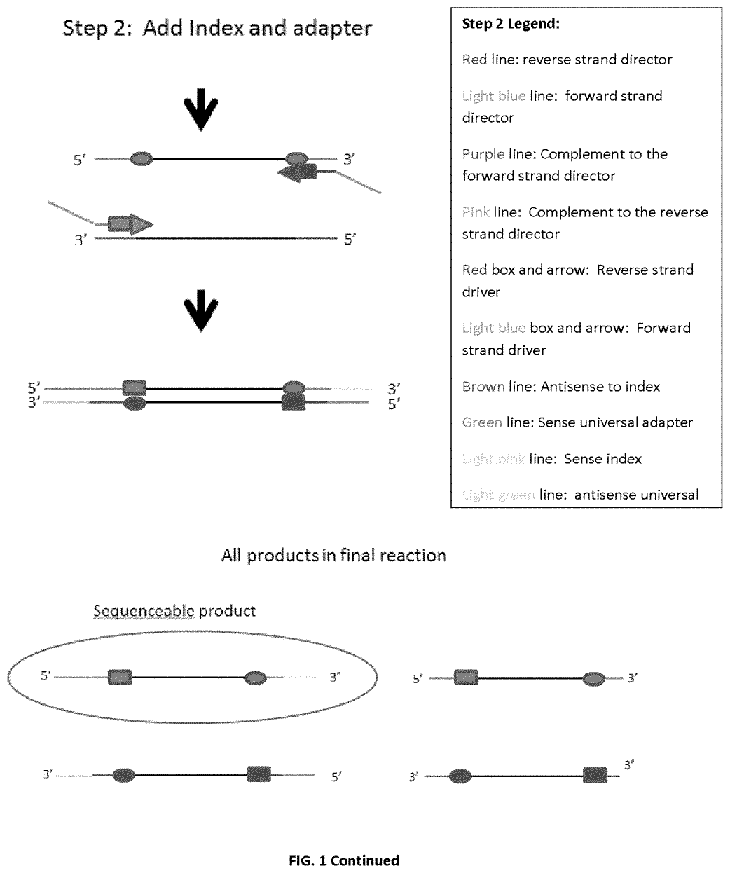

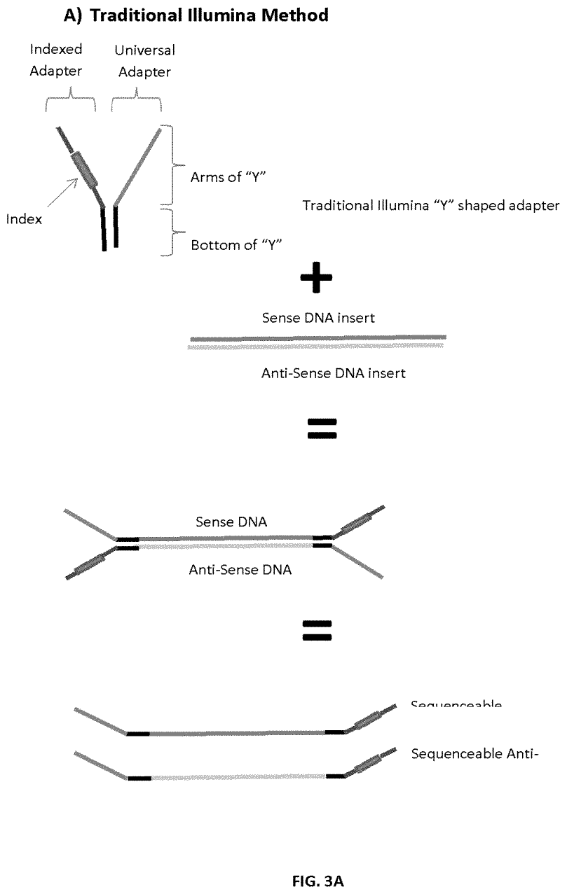

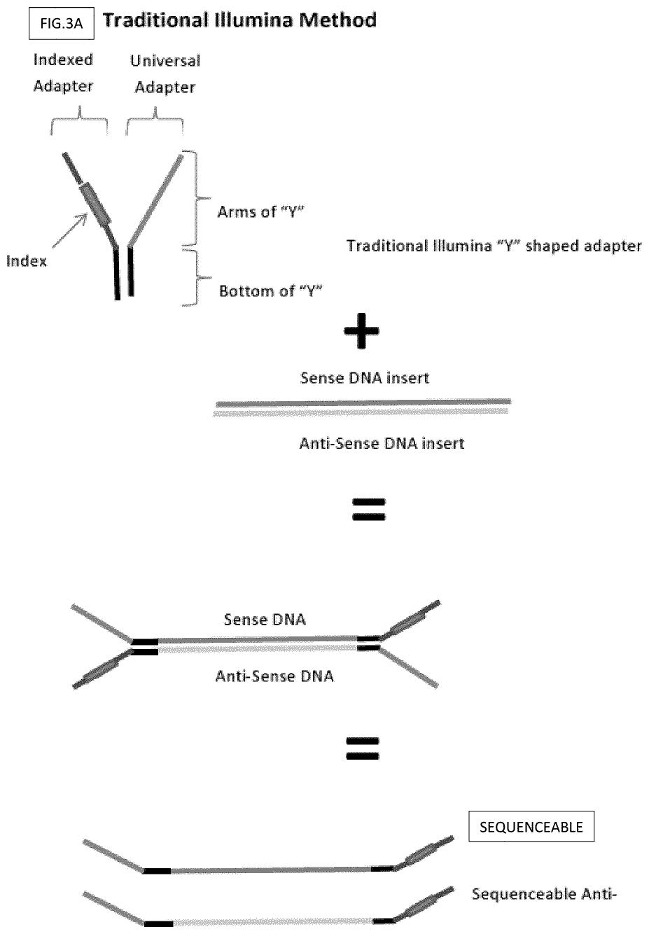

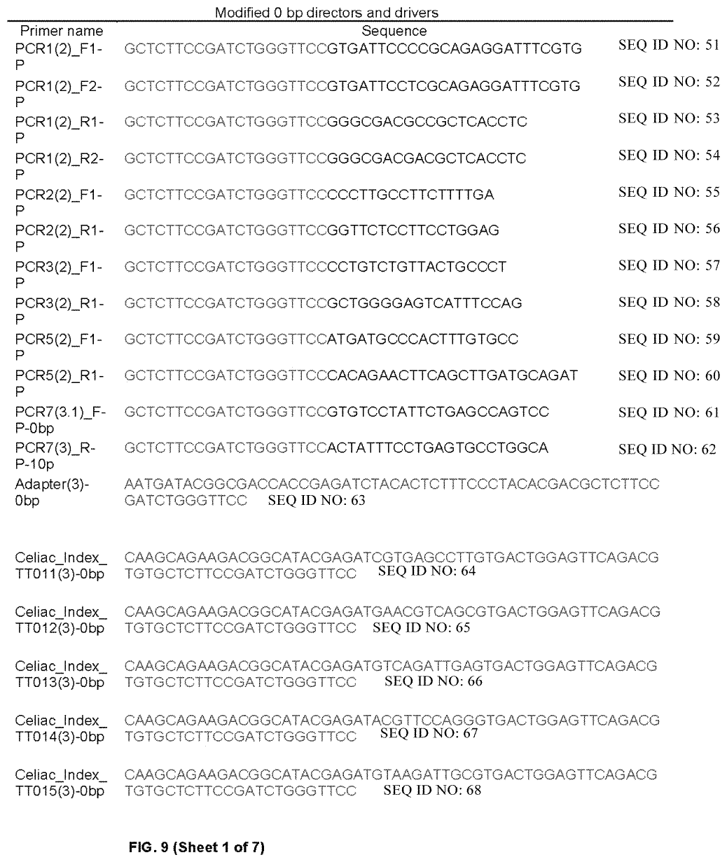

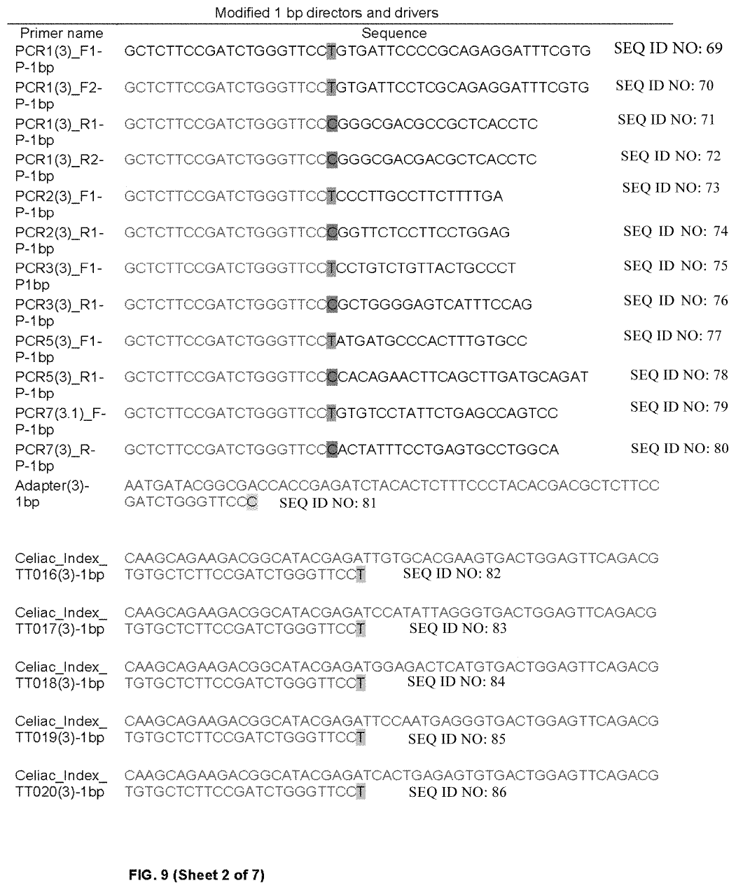

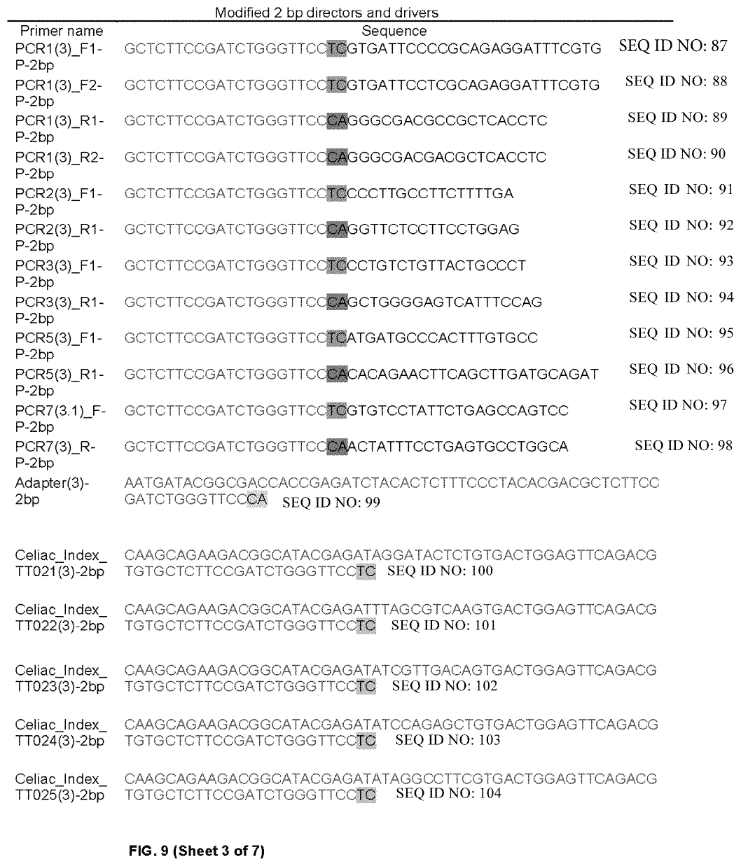

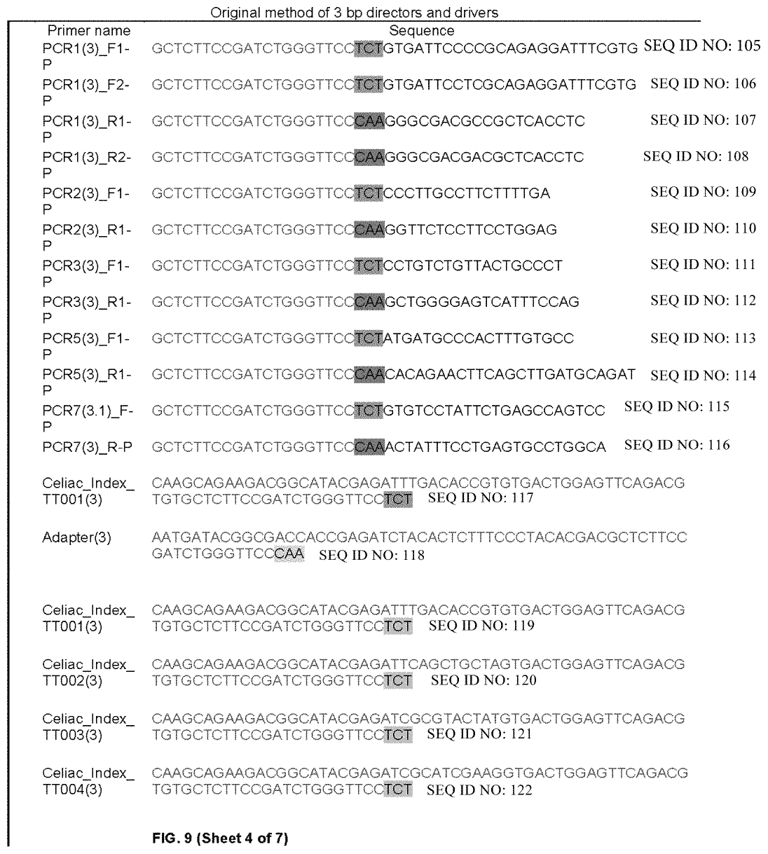

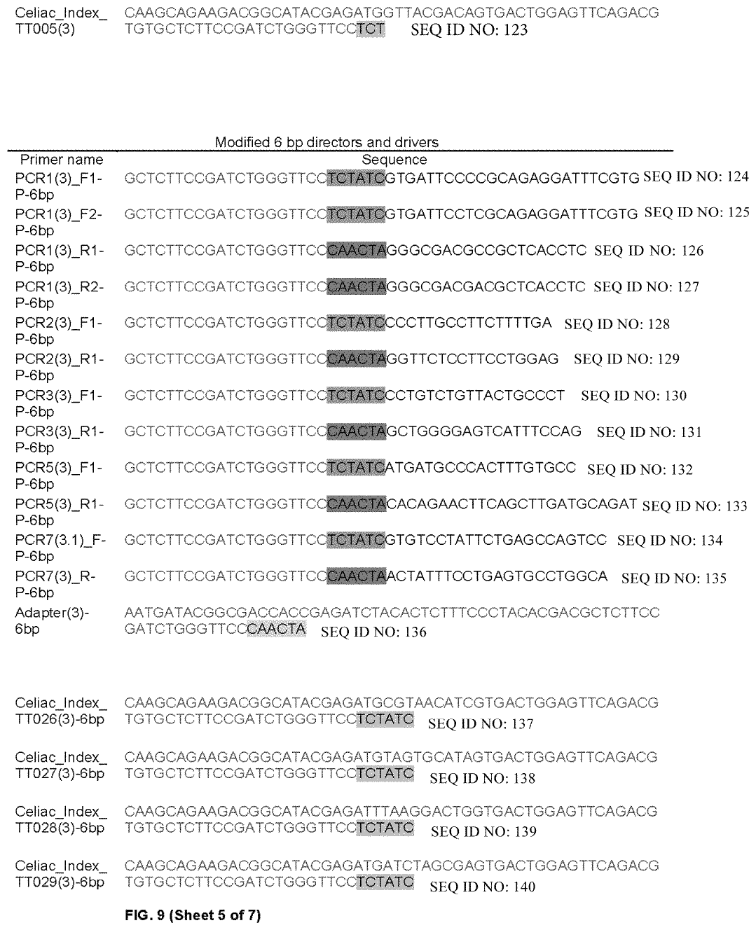

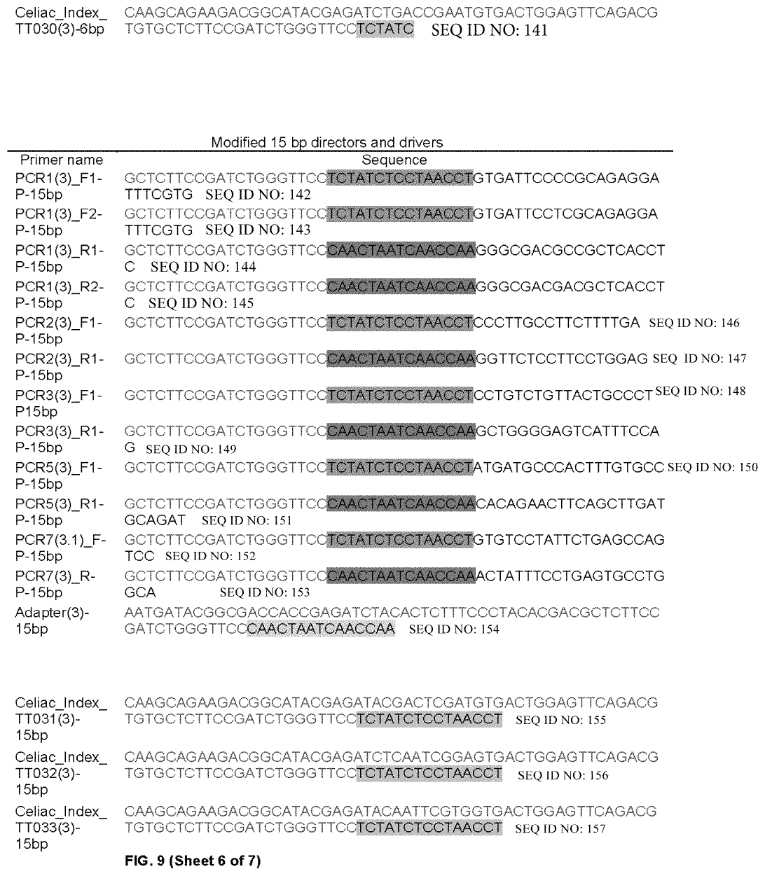

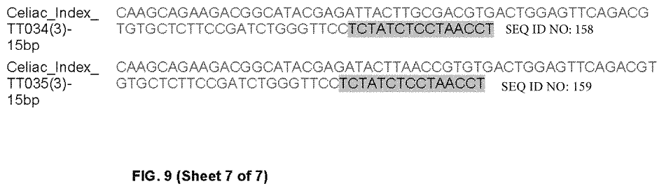

“Director” refers to at least one nucleotide that is inserted within a PCR forward primer and/or reverse primer, and that is not complementary, and therefore does not hybridize with, a target polynucleotide that is desired to be amplified using the PCR forward and reverse primers, which are complementary to portions of the target polynucleotide, and which contain an insertion of the director. Directors include a single nucleotide (referred to as “director nucleotide”) as well as nucleotide sequence (referred to as “director nucleotide sequence” or “director sequence”) of at least one nucleotide. The nature of the nucleotide and/or nucleotides in the director nucleotide and in the director sequence is not limited to any particular nucleotide and/or sequence, so long the director nucleotide and/or sequence is not complementary, and therefore do not hybridize with, the target polynucleotide in the vicinity of the forward primers and reverse primers that contain the director, and that are used for amplification of the target polynucleotide. It may be desirable to design directors to avoid secondary structures such as hairpins, homodimers, and heterodimers, and avoid amplifying sequences that are similar to the target polynucleotide. Directors may be double-stranded, or single-stranded such as reverse strand director sequences and forward strand director sequences produced by PCR amplification (FIGS. 1 and 3B). Directors may be from 1 to 30 base pairs (bp), including from 1 to 29 bp, 1 to 28 bp, 1 to 27 bp, 1 to 26 bp, 1 to 25 bp, 1 to 24 bp, 1 to 23 bp, 1 to 22 bp, 1 to 21 bp, 1 to 20 bp, 1 to 19 bp, 1 to 18 bp, 1 to 17 bp, 1 to 16 bp, 1 to 15 bp, 1 to 14 bp, 1 to 13 bp, 1 to 12 bp, 1 to 11 bp, 1 to 10 bp, 1 to 9 bp, 1 to 8 bp, 1 to 7 bp, 1 to 6 bp, 1 to 5 bp, 1 to 4 bp, 1 to 3 bp, and 1 to 2 bp. Directors are exemplified by, but not limited to, the 1, 2, 3, 6, and 15 bp sequences of FIGS. 5 and 9.

“Driver” refers to at least one nucleotide that is equal in length to, and hybridizes with, the complementary strand generated by PCR amplification of a director. Drivers include a single nucleotide (referred to as “driver nucleotide”) as well as nucleotide sequence (referred to as “driver nucleotide sequence” or “driver sequence”) of at least two nucleotides. Drivers may be double-stranded, or single-stranded such as reverse strand driver sequences and forward strand driver sequences produced by PCR amplification (FIGS. 1 and 3B). Drivers may be from 1 to 30 base pairs (bp), including from 1 to 30 bp, 1 to 29 bp, 1 to 28 bp, 1 to 27 bp, 1 to 26 bp, 1 to 25 bp, 1 to 24 bp, 1 to 23 bp, 1 to 22 bp, 1 to 21 bp, 1 to 20 bp, 1 to 19 bp, 1 to 18 bp, 1 to 17 bp, 1 to 16 bp, 1 to 15 bp, 1 to 14 bp, 1 to 13 bp, 1 to 12 bp, 1 to 11 bp, 1 to 10 bp, 1 to 9 bp, 1 to 8 bp, 1 to 7 bp, 1 to 6 bp, 1 to 5 bp, 1 to 4 bp, 1 to 3 bp, and 1 to 2 bp. Drivers are exemplified by the 1, 2, 3, 6, and 15 bp sequences that have the same sequence as, or that are complementary to, director sequences. Data herein (Example 3) show that longer driver sequence lengths result in higher efficiency of the invention's methods. In one embodiment, a driver is added to the 3′ end of a universal sequence and/or to the 3′ end of an index sequence (FIG. 1B).

“Index” and “indexed” polynucleotide sequence means a unique nucleotide sequence that is distinguishable from the sequence of other indices as well as the sequence of other nucleotide sequences within polynucleotides contained within a sample. An index sequence is useful as a barcode where different members of the same molecular species can contain the same index sequence and where different species within a population of different polynucleotides can have different unique indices. An index sequence can be a random or a specifically designed nucleotide sequence. An index sequence can be of any desired sequence length so long as it is of sufficient length to be a unique nucleotide sequence within a plurality of indices in a population and/or within a plurality of polynucleotides that are being analyzed or interrogated. A nucleotide index is useful, for example, to be attached to a target polynucleotide to tag or mark a particular species for identifying all members of the tagged species within a population. Methods for designing and making index sequences are known in the art (Illumina TruSeq Adapters Demystified Rev. A, © 2011 Tufts University Core Facility), and U.S. Pat. No. 9,926,598. Index polynucleotide sequences are exemplified in FIGS. 5, 6, and 9.

“Universal polynucleotide sequence” is a sequence that enables amplification of any target polynucleotide of known or unknown sequence that has been modified to enable amplification with the universal primers. In one embodiment, such amplification produces an amplified target polynucleotide containing a “universal” sequence, such as a universal adapter sequence, at the target polynucleotides' 3′ and/or 5′ ends. The attachment of universal known ends to a library of DNA fragments by ligation allows the amplification of a large variety of different sequences in a single amplification reaction. The sequences of the known sequence portion of the nucleic acid template can be designed such that type 2s restriction enzymes bind to the known region, and cut into the unknown region of the amplified template. Universal primers are known in the art and exemplified by Illumina's Sequences S1 and S2 which, in combination, direct amplification of a template by solid-phase bridging amplification reaction. The template to be amplified must itself comprise (when viewed as a single strand) at the 3′ end a sequence capable of hybridizing to sequence S1 in the forward primers and at the 5′ end a sequence the complement of which is capable of hybridizing to sequence S2 the reverse primer. Methods for designing and making universal sequences are known in the art (Illumina TruSeq Adapters Demystified Rev. A, CO 2011 Tufts University Core Facility), and U.S. Pat. No. 8,765,381. Universal polynucleotide sequences are exemplified in FIG. 9.

“Adapter” or “adaptor” or a “linker” is a short, chemically synthesized, single-stranded or double-stranded oligonucleotide that can be ligated to the 3′ and/or 5′ ends of other DNA or RNA molecules. Adapters containing specific sequences designed to interact with next-generation-sequencing (NGC) platforms (such as the surface of the flow-cell or beads may be ligated to one or both of the 3′ and 5′ ends of target polynucleotides prior to sequencing. For example, adapters include “indexed adapters” and “universal adapters.” The primary function of both indexed adapters and universal adapters is to allow any DNA sequence to bind to a flowcell for next generation sequencing (NGS), and to allow for PCR enrichment of only adapter ligated DNA sequences for cluster generation (such as either on a MiSeq flowcell or on an Ion Torrent bead). Next generation sequencing (NGS) does not require indexed adapters and could be done exclusively with universal adapters. However, doing so would limit any run to only one sample. The addition of indexes unique to each sample allows for the mixing of two or more samples, for sequencing to occur, and for results to be analyzed after the sequencing is complete. The structure of adapters is dictated by the sequencing platform.

“Indexed adapters” (also referred to as “index adapters”) contain index polynucleotide sequences, and are known in the art as exemplified by TruSeq Indexed Adapter: 5′ P*GATCGGAAGAGCACACGTCTGAACTCCAGTCACATCTCGTATGCC 3′ (SEQ ID NO: 1) Indexed adapters allow for indexing or “barcoding” of samples so multiple DNA libraries can be mixed together into one sequencing lane (known as multiplexing). Methods for designing and making index adapters are known in the art (Illumina TruSeq Adapters Demystified Rev. A, © 2011 Tufts University Core Facility).

“Universal adapters” contain universal polynucleotide sequences, and are known in the art as exemplified by TruSeq Universal Adapter: 5′AATGATACGGCGACCACCGAGATCTACACTCTTTCCCTACACGACGCTCTTCCGATC*T 3′. (SEQ ID NO: 2). The stars (*) in the above TruSeq Indexed Adapter and TruSeq Universal Adapter indicate a phosphorothioate bond between the last C and T to prevent cleaving off the last T that is needed for annealing the overhang. The phosphate group on the indexed adapter is required to ligate the adapter to the DNA fragment. The NNNNNN in the above exemplary TruSeq Indexed Adapter represents the “barcode.” The last 12 bases are complementary if the Indexed Adapter is reversed. Methods for designing and making universal adapters are known in the art (Illumina TruSeq Adapters Demystified Rev. A, © 2011 Tufts University Core Facility).

A “primer” sequence is a short single-stranded DNA that hybridizes to a target polynucleotide sequence, and serves as a starting point for synthesis of a complementary strand of the target polynucleotide sequence. “PCR primer” is a primer used in a “polymerase chain reaction” (“PCR”). Design principles for PCR primers are known in the art, including primer length, specificity to the target polynucleotide sequence, melting temperature (Tₘ) value, annealing temperature (Tₐ), freedom of strong secondary structures and self-complementarity, and GC content.

“Target specific” and “site specific” when used in reference to a primer or other oligonucleotide sequence is intended to mean a primer or other oligonucleotide sequence that includes a nucleotide sequence that is complementary to, and that specifically and selectively hybridizes (i.e., anneals) to, at least a portion of a target polynucleotide sequence. Target specific primers include forward and reverse primers, universal primers, index primers, sequencing primers, and the like.

“Forward primer” is a primer sequence that hybridizes to the sense strand of a DNA sequence of interest. In contrast, a “reverse primer” hybridizes to the anti-sense strand of the DNA sequence of interest.

“Universal primer” sequences refer to a primer sequence that is complementary to, and that specifically and selectively hybridizes (i.e., anneals) to, a universal polynucleotide sequence.

“Index primer” and “indexed primer” sequences interchangeably refer to a primer sequence that is complementary to, and that specifically and selectively hybridizes (i.e., anneals) to, an index polynucleotide sequence.

“Insert,” “insertion,” and grammatical equivalents refer to a change in a polynucleotide sequence that results in the addition of one or more nucleotides. For example, a PCR primer that is complementary along its entire length to a region of a target polynucleotide sequences may be modified by insertion of a director (e.g., director nucleotide or director sequence) that is not complementary to the region of a target polynucleotide, and the presence of the director within the modified PCR primer therefore results in hybridization of only a portion (located at either the 3′ end or 5′ end of the inserted director) of the modified PCR primer to the region of the target polynucleotide sequences.

A “sense strand” and “coding strand” interchangeably refer to a segment within double-stranded DNA that runs from 5′ to 3′, and that is complementary to the “antisense strand” (i.e., “template strand”) of DNA, which runs from 3′ to 5′.

“Complement” and “complementary” when in reference to a sequence of interest interchangeably refer to a nucleic acid sequence that can form a double-stranded structure with the sequence of interest by matching base pairs. For example, the complementary sequence to 5′-G-T-A-C-3′ is 3′-C-A-T-G-5′. In one embodiment, PCR primers are 100% complementary along their entire length to a region of a target polynucleotide. In another embodiment, it may be desirable to modify a PCR primer that is complementary along its entire length to a region of a target polynucleotide sequences may be modified by insertion of a director (e.g., director nucleotide or director sequence) that is not complementary to the region of a target polynucleotide, and the presence of the director within the modified PCR primer therefore results in complementarity of only a portion (located at either the 3′ end or 5′ end of the inserted director) of the modified PCR primer to the region of the target polynucleotide sequences.

“Amplification” refers to making copies of polynucleotide sequences of interest. Amplification methods include both thermocycling (such as “polymerase chain reaction” (“PCR”)) amplification) and isothermal amplifications (such as described in application number WO07107710), using a commercially available Solexa/Illumina cluster station as described in PCT/US/2007/014649. The cluster station is essentially a hotplate and a fluidics system for controlled delivery of reagents to a flowcell.

“Amplicon” is a nucleotide sequence that is the source and/or product of amplification or replication events. It can be formed artificially, using various methods including polymerase chain reactions (PCR) or ligase chain reactions (LCR), or naturally through gene duplication.

“Variable” sequence refers to a segment of a chromosome characterized by variation in the number of tandem repeats at one or more loci. In some embodiments, a “variable” sequence is a “hypervariable” sequence, which refers to a segment of a chromosome characterized by considerable variation in the number of tandem repeats at one or more loci. Repeats in the hypervariable region are highly polymorphic. A hypervariable locus refers to a locus with many alleles; especially those whose variation is due to variable numbers of tandem repeats. A hypervariable region (HVR) refers to a chromosomal segment characterized by multiple alleles within a population for a single genetic locus.

“Polymerase chain reaction “(“PCR”) is a method for making copies of a specific DNA segment using repeated thermal PCR cycles. “PCR cycle” refers to a combination of denaturing a double-stranded template DNA by heating to separate it into two single strands, annealing the DNA primers to the template DNA by lowering the temperature, and extending the new DNA strand by the Taq polymerase enzyme and by raising the temperature.

“Hybridizing” and grammatical equivalents refer to a process by which single-stranded DNA or RNA molecules anneal to complementary single-stranded DNA or RNA through base pairing. Hybridization and the strength of hybridization (i.e., the strength of the association between the nucleic acids) is impacted by such factors as the degree of complementarity between the nucleic acids, stringency of the conditions involved, the melting temperature (Tm) of the formed hybrid, and the G:C ratio within the nucleic acids. Conditions for hybridizing DNA molecules, such as primers and target DNA polynucleotides are known in the art, and exemplified herein.

“Operable combination” and “operably linked” when in reference to the relationship between nucleic acid sequences refers to fusing the sequences in frame such that they perform their intended function. For example, operably linking a promoter sequence to a nucleotide sequence of interest refers to fusing the promoter sequence and the nucleotide sequence of interest in a manner such that the promoter sequence is capable of directing the transcription of the nucleotide sequence of interest and/or the synthesis of a polypeptide encoded by the nucleotide sequence of interest.

“Fuse,” “fusion,” and grammatical equivalents when made in reference to a first and second nucleotide sequences refer to the linkage of the first and second nucleotide sequences via phosphodiester bonds. Fusion of a first and second nucleotide sequences may be direct or indirect. “Direct” fusion refers to the absence of intervening nucleotides between the first and second nucleotide sequences. “Indirect” fusion refers to the presence of one or more nucleotides between the first and second nucleotide sequences. For example, the term “index sequence fused at its 3′ end to a driver” refers to an index sequence that is fused, directly or indirectly, at its 3′ end to a driver.

The terms “3′ end” and “5′ end” when in reference to a nucleotide sequence refer to the terminal nucleotide base that is located at, respectively, the 3′ terminal and 5′ terminal of the nucleotide sequence.

The terms “3′ terminal region” and “5′ terminal region” when in reference to a nucleotide sequence refer to a portion of the nucleotide sequence that is approximately a third of the length of the nucleotide sequence and that spans and includes, respectively, the 3′ end and 5′ end the nucleotide sequence.

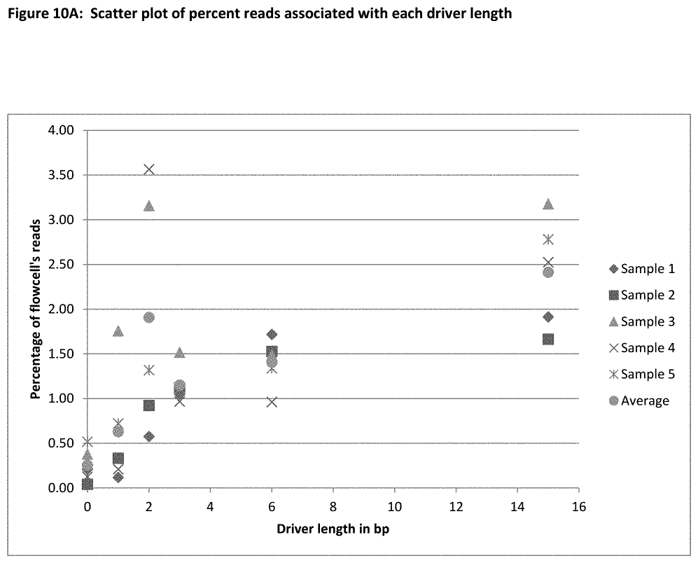

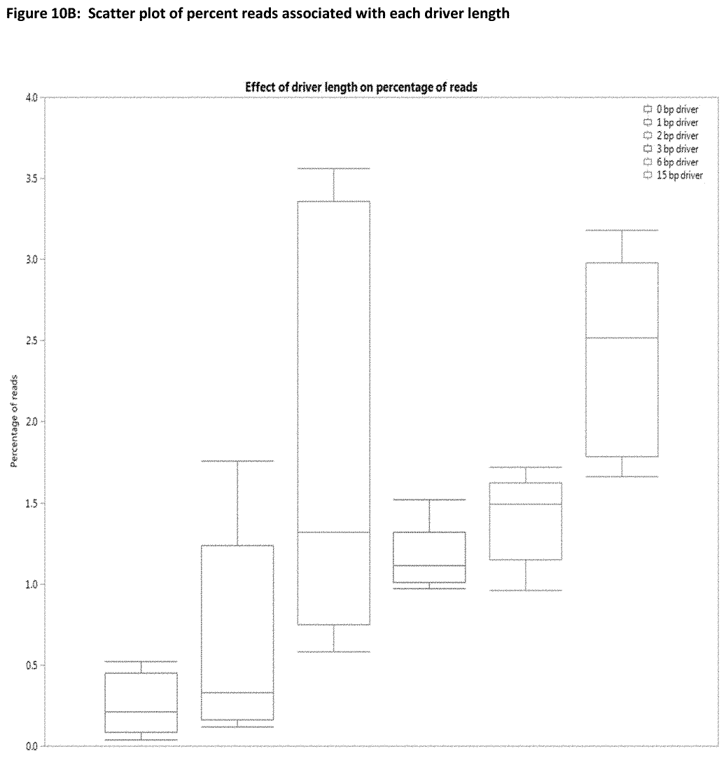

“Efficiency” when in reference to an amplicon refers to the percentage of total reads of the amplicon. “Higher efficiency” refers to an increase in the percentage of total reads, exemplified by an increase of at least 0.1 fold (i.e., 10%), including an increase of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 fold, and exemplified by an increase from 0.1 fold (i.e., 10%) fold to 100 fold (i.e., 10,000 fold), including from 0.1 to 90 fold, 1 to 90 fold, 1 to 80 fold, 1 to 70 fold, 1 to 60 fold, 1 to 50 fold, 1 to 40 fold, 1 to 30 fold, 1 to 29 fold, 1 to 28 fold, 1 to 27 fold, 1 to 26 fold, 1 to 25 fold, 1 to 24 fold, 1 to 23 fold, 1 to 22 fold, 1 to 21 fold, 1 to 20 fold, 1 to 19 fold, 1 to 18 fold, 1 to 17 fold, 1 to 16 fold, 1 to 15 fold, 1 to 14 fold, 1 to 13 fold, 1 to 12 fold, 1 to 11 fold, 1 to 10 fold, 1 to 9 fold, 1 to 8 fold, 1 to 7 fold, 1 to 6 fold, 1 to 5 fold, 1 to 4 fold, 1 to 3 fold, and 1 to 2 fold, as exemplified by a 27 fold (i.e. 2,700%) increase shown in Example 3, Table 3, and FIG. 10A-B.

The terms “higher,” “greater,” and grammatical equivalents (including “increase,” “elevate,” “raise,” etc.) when in reference to the level of any molecule (e.g., amplicon, nucleic acid sequence, amino acid sequence, etc.) and/or phenomenon (e.g., amplification of a nucleotide sequence, expression of a gene, etc.), specificity of binding of two molecules (e.g., binding of a director to a driver), in a first sample relative to a second sample, mean that the quantity of the molecule and/or phenomenon in the first sample is higher than in the second sample by any amount that is statistically significant using any art-accepted statistical method of analysis.

“Kit” is used in reference to a combination of reagents and other materials. A kit may include reagents such as buffering agents, nucleic acid stabilizing reagents, protein stabilizing reagents, and/or testing containers. In one embodiment, the kit further comprises instructions for using the reagents, such as for amplification of a target polynucleotide sequence, exemplified by, but not limited to, instruction in Example 2.

### DESCRIPTION OF THE INVENTION

The invention provides compositions and methods for accurately and specifically amplifying sequences to allow for accurate and specific detection of mutations, such as in disease-associated genes and alleles, thus distinguishing true nucleotide variants over random nucleotide sequencing errors. In one embodiment, this is accomplished, prior to sequencing, by using a combination of director and driver in a combination of two PCR reactions. Thus, in one embodiment, this is accomplished by first amplifying a nucleotide sequence of interest to introduce a director that creates a specific target for a second amplification in which a driver, which specifically hybridizes to the director, drives the directionality of the second amplification. The amplification produces an amplicon of a sense or antisense strand of a double-stranded nucleotide sequence of interest. The amplicon may optionally contain universal sequences and/or index sequences that facilitate subsequent sequencing of the amplicon, such as using sequencing-by-synthesis. The reagents of the first and second amplification steps may be combined in a single reaction mixture.

Thus, in one embodiment, the invention's methods include two steps that, optionally, occur together in the same reaction vessel in order. First, a PCR occurs where the PCR primers include a director that creates a specific target. Second, the universal adapter and optional index adapter are added using primers that include a driver that specifically binds to the director. If the index adapter's driver finds the index adapter's specific director it will base pair and extend. If the index adapter's driver finds the universal adapter's specific director it will not base pair and will not extend. Conversely, if the universal adapter's driver finds the universal adapter's specific director it will base pair and extend. If the universal adapter's driver finds the index adapter's specific director it will not base pair and will not extend. Without the specificity of the combination of a driver and director the methods are very inefficient.

In one embodiment, the forward PCR primers 1 (director 1) and 3 (driver 1) directly incorporate a director/driver to the sense strand. The reverse PCR primers 2 (director 2) and 4 (driver 2) directly incorporate a director/driver to the antisense strand. Through PCR, the sense strand is copied as a complementary antisense strand and the antisense strand is copied as a complementary sense strand. As a final product, the sense strand ultimately has a direct 5′ incorporation of the PCR 1 and 3 driver/director and an indirect 3′ incorporation of the complement of the PCR 2 and 4 driver/director. As a final product, the antisense strand ultimately has a direct 5′ incorporation of the PCR 2 and 4 driver/director and an indirect 3′ incorporation of the complement of the PCR 1 and 3 driver/director. Because director 1 and driver 1 have the same sequence (and director 2 has the same sequence as driver 2) the driver does not directly hybridize with the director. Instead, it hybridizes with the transcribed, complementary strand produced as primer 1 is amplified. The first director of the first primer and second driver of the second primer are unique from one another. They cannot be equal and cannot be complementary to one another. Their uniqueness from one another is what creates specificity for the second PCR and is what gives directionality to the system.

One advantage of the invention's methods is that they produce sequenceable amplicons at a higher efficiency compared to amplification methods that omit using the invention's combination of director and driver. For example, while the total amplicon count is similar without the use of the invention's combination of driver and director, however, the majority of products cannot be sequenced or otherwise interfere with sequencing of the sequenceable amplicon.

A further advantage of the invention's methods is that they accurately and specifically produce amplicons, thus enabling accurate and specific sequencing and detection of variable and hypervariable alleles. For example, genomic sequences amplified using the invention's methods and compositions may be subjected to sequencing, thus enabling diagnosis of disease. In one embodiment, the invention's methods produced sequences that successfully diagnosed patients as having celiac disease by confirming a patient as having DQB1:03/DQB1:06 alleles, and another patient having DQA1:02/DQA1:01:03 alleles (Example 2), which are different from the types that are traditionally tested.

Another advantage of the invention's methods is that their use to detect particular genes does not require amplifying and sequencing an entire disease-associated gene (such as celiac disease HLA DQA and HLA DQB genes), but may be accomplished by amplifying and sequencing only the hypervariable regions that define the specific disease-associated alleles. NGS carries strand level information and all variants found within a strand can be understood in a cis/trans context. While not sufficient by SSO or even Sanger, by using a few targeted regions of, for example, less than 300 bp, it is contemplated that alleles (such as celiac disease HLA DQA and HLA DQB alleles) can be determined down to the second or third field.

Yet another advantage of the invention's methods is that they can be performed by combining the reagents for more than one PCR step in a single reaction vessel, thereby reducing time for technician involvement, reducing the likelihood for transcription errors, and reducing the likelihood for secondary PCR contamination.

An additional advantage of the invention's methods is that they use lower primer concentrations and fewer amplification cycles to drive amplification reactions to completion, thus reducing cost and time.

One characteristic of the invention's methods is that they generate an amplicon of either a sense strand or antisense strand of the target polynucleotide. The generated amplicon is fused to a universal adapter and index adapter, and is thus complementary to the flowcell surface and are retained during the cluster generation step for subsequent sequencing (FIG. 3B). This is in contrast to conventional library preparation methods that use Illumina Y shaped adapters (FIG. 3A) that allow for both the sense strand and antisense strand to create reads in which both the sense and antisense strands of the target polynucleotide have a universal adapter and index adapter, and both strands are subsequently sequenced.

In some embodiments, the invention's methods target a selected region of a gene with sufficient read depth, such that data provided by one strand (such as the sense strand or antisense strand, and more preferably the sense strand) that is generated by the invention's methods provides adequate information regarding the actual sequence. In some embodiments, the range of read depths is dependent on instrument, assay complexity, and number of samples or runs. As an example, 40,000 samples could be run on one MiSeq run and still get 100 fold coverage. Even without full optimization, depths measured are in the 2,000-20,000 range. In one embodiment, the read depth is from 20 to 1,000, including from 20 to 900, 20 to 800, 20 to 700, 20 to 600, 20 to 500, 20 to 400, 20 to 300, 20 to 200, 20 to 100, and 20 to 50. For example, in some embodiment, the read length is 100 for germline variant samples, and at least 500 for somatic (such as cancer) variant samples.

The invention's methods were developed and applied to the exemplary HLA genes associated with celiac disease. Celiac disease is a long-term autoimmune disorder that primarily affects the small intestine. Celiac disease is “permissive” with the right HLA type. Without the right HLA type celiac disease doesn't occur, but with the right type it can occur. HLA Class II molecules are heterodimers containing an alpha and beta chain, with each chain encoded by a different gene. There are several different HLA Class II molecules including HLA-DP, HLA-DQ, and HLA-DR, as well as many different pseudogenes. All HLA genes including both Class I and Class II are encoded on chromosome 6.

Current genetic testing for celiac disease can rule out celiac disease, can indicate an individual is at risk to develop celiac disease, but cannot, alone, directly diagnose celiac disease. Almost all people (95%) with celiac disease have either the variant HLA-DQ2 allele or (less commonly) the HLA-DQ8 allele. However, about 20-30% of people without celiac disease have also inherited either of these alleles. There are three different celiac disease permissive HLA-DQ types, either DQ2.5, DQ2.2, or DQ8 (in order of celiac disease-associated frequency). This nomenclature indicates a heterodimer containing specific alpha and beta chains. DQ2.5 contains DQB1*0201 and DQA1*0501. DQ2.2 contains DQB1*0202 and DQA1*0201. DQ8 contains DQB1*0302 and DQA1*0301. Traditionally, genetic testing for celiac disease aims to detect the presence of the following alleles:

TABLE 1

| | DQB1 | DQA1 |
|---|---|---|
| Type | allele | allele |
| DQ2.5 | 02:01 | 05:01 |
| DQ2.2 | 02:02 | 02:01 |
| DQ8 | 03:02 | 03:01 |

Additionally, current celiac disease testing reports HLA-DQ typing at a low resolution for all patients, not just celiac disease permissive patients. While on the one hand clinicians need to confirm whether any of the above types are present, there is still a desire to report types not on this list. This is one reason why there are six PCR products, more than necessary to prove a simple positive/negative result, in the invention's assay design.

Initial attempts in developing the invention's methods and compositions were not completely optimized. One initial strategy in developing the invention's methods (Example 1) attempted to sequence a few targeted regions of less than 300 bp of the hypervariable regions that define specific alleles in the celiac disease HLA DQA and HLA DQB genes, by using universal tags to randomly add the index adapter or universal adapter to either the forward or reverse strand. However, this strategy did not succeed (Example 1).

The invention's subsequent successful methods for amplification of an exemplary sense strand of a double-stranded target polynucleotide are exemplified herein in Examples 2 and 3, FIGS. 1, 2, and 3B). One characteristic of the invention's methods is that they create either a sense strand or antisense strand of the target polynucleotide, which is optionally fused to a universal adapter thus producing complementarity to the flowcell surface and retention during the cluster generation step for subsequent sequencing. The remaining generated sequences are not complementary to the flowcell surface and are washed away during the cluster generation step for subsequent sequencing.

The invention's methods may be used for amplification of only a sense strand or only an antisense strand of a double-stranded target polynucleotide sequence into a sequenceable sequence. Thus, in one embodiment, the invention provides a method for amplifying a target polynucleotide sequence, comprising two amplification steps.

The first amplification comprises contacting a double-stranded target polynucleotide sequence with i) a first PCR primer that is modified by having an insertion of a director, and ii) a second primer modified by having an insertion of a second director, the contacting is under conditions sufficient for amplifying the double-stranded target polynucleotide sequence to produce a first plurality of amplicons comprising a first single-stranded amplicon that comprises at least one single strand of the double-stranded target polynucleotide sequence, the at least one single strand having an insertion of the director in either its 3′ or 5′ terminal regions and an insertion of the complement of the second director in its other terminal region.

The second amplification step comprises contacting the at least one single strand with i) a third primer fused at its 3′ end to a first driver, and ii) a fourth primer fused at its 3′ end to a second driver, wherein the first has the same sequence as the first director, and the second driver has the same sequence as the second director, wherein the 5′ end of either the first driver or the second driver is fused to a universal adapter sequence, and wherein the contacting is under conditions sufficient for amplifying the at least one single strand to produce a second plurality of amplicons comprising a second single-stranded amplicon having the universal adapter sequence fused to its 5′ end, and comprising at its 3′ end either the at least one single strand containing an insertion of the director in either its 3′ or 5′ terminal regions, and containing an insertion of the complement of the director in its other terminal region.

In one embodiment, it may be desirable to carry out the two amplification steps in a single reaction mixture.

In some embodiments, where it may be desirable to amplify a sense strand of double-stranded target polynucleotide sequence, the first primer and the third primer are forward primers, and the second primer and the fourth primer are reverse primers (FIGS. 1, 2, and 3B).

In some embodiments, where it may be desirable to amplify an antisense strand of double-stranded target polynucleotide sequence, the first primer and the third primer are reverse primers, and the second primer and the fourth primer are forward primers.

In one embodiment, where the sense strand of double-stranded target polynucleotide sequence is amplified, the invention's method generates a second single-stranded amplicon that contains, fused in operable combination from the 5′ end to the 3′ end, a) the universal adapter sequence, and b) the at least one single strand having an insertion of the director in either its 3′ or 5′ terminal regions and an insertion of the complement of the director in its other terminal region.

In a particular embodiment, where it is desirable to include an index sequence in the amplified sense strand, the 5′ end of the first driver of the third primer is fused to a unique index sequence, and the 5′ end of the second driver of the fourth primer is fused to the universal adapter sequence. This produces a second single-stranded amplicon fused at its 3′ end to the unique index sequence, and fused at its 5′ end to the universal adapter sequence (FIG. 1). Thus, the second single-stranded amplicon contains an insertion in its 3′ terminal region of a sense strand of the unique index sequence, and an insertion in its 5′ terminal region of a sense strand of the universal adapter sequence. In a particular embodiment, the second single-stranded amplicon contains, fused in operable combination from the 5′ end to the 3′ end, a) the universal adapter sequence, b) the at least one single strand having an insertion of the director in either its 3′ or 5′ terminal regions and an insertion of the complement of the director in its other terminal region, and c) the unique index sequence. In a particular embodiment, the unique index sequence is comprised in an index adapter.

In a particular embodiment, where it is desirable to include an index sequence in the amplified antisense strand, the 5′ end of the first driver of the third primer is fused to the universal adapter sequence, and the 5′ end of the second driver of the fourth primer is fused to a unique index sequence. In a particular embodiment, the second single-stranded amplicon is fused at its 5′ end to the unique index sequence, and fused at its 3′ end to the universal adapter sequence. In a further embodiment, the second single-stranded amplicon contains an insertion in its 5′ terminal region of a sense strand of the unique index sequence, and an insertion in its 3′ terminal region of a sense strand of the universal adapter sequence. In a particular embodiment, the unique index sequence is comprised in an index adapter.

While not necessary, it may be desirable to contact the reagents of the first and second amplification steps in a single reaction mixture (Example 2 and 3).

It may be desirable to sequence the single-stranded amplicon comprised in the second plurality of amplicons, such as using sequencing-by-synthesis (SBS), using methods known in the art (U.S. Pat. No. 8,765,381).

On advantage of the invention's methods is that they produce the second single-stranded amplicon at a higher efficiency than in the absence of one or more of the director, the first driver, and the second driver. Data herein demonstrate that inclusion of the director and driver increased the efficiency by 27 fold (i.e. 2,700%) (Example 3, Table 3, and FIG. 10A-B).

Another advantage of the invention's methods is that they may be accomplished using a lower concentration of one or both of the first primer and the first primer than the concentration of one or both of the second primer and the second primer. The concentration of one or both of the first primer and the second primer is 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, and 10% or less than the concentration of one or both of the third primer and the fourth primer, including from 10% to 25% less, 10% to 24% or less, 10% to 23% or less, 10% to 22% or less, 10% to 21% or less, 10% to 20% or less, 10% to 19% or less, 10% to 18% or less, 10% to 17% or less, 10% to 16% or less, and 10% to 15% or less than the concentration of one or both of the third primer and the fourth primer. Data herein demonstrate the successful use of ⅙^{th} (i.e., 16%) the concentration of the site specific PCR primer, compared to the concentration of either the index primer or universal primer, to exhaust reagents and drive the PCR reactions to completion.

A further advantage of the invention's methods is that they may be accomplished using fewer than 100 cycles, including fewer than each of 90, 80, 70, 60, 50, 45, 40, 35, 30, 25, and 20 cycles. Data herein demonstrate successful use of 50 PCR cycles (Examples 2 and 3).

In a particular embodiment, the target polynucleotide sequence comprises genomic DNA, exemplified by, but not limited to, the DQB1 gene and/or DQA1 gene. In a particular embodiment, the genomic DNA is not fragmented. In a further embodiment, the genomic DNA comprises a variable and/or hypervariable sequence of an allele. While not intending to limit the invention to any particular variable and/or hypervariable allele, in one embodiment, the hypervariable allele comprises one or more of DQ2.5 allele, DQ2.2 allele, DQ8 allele, DQB1 02:01 allele, DQB1 02:02 allele, DQB1 03:02 allele, DQA1 05:01 allele, DQA1 02:01 allele, and DQA1 03:01 allele (Table 1). In one embodiment, the hypervariable allele comprises one or more of KIR2DL1 allele, KIR2DL2 allele, KIR2DL3 allele, and KIR2DL4 allele. In one embodiment, hypervariable alleles comprise one or more of. In one embodiment, variable alleles include CYPD6*2 allele, CYPD6*3 allele, and CYPD6*4 allele.

The invention's methods may be used for amplification of a sense strand or an antisense strand of a target polynucleotide. Thus, in one embodiment, the invention provides a method for amplifying target polynucleotide sequences, comprising, contacting i) a sample comprising a plurality of double-stranded target polynucleotide sequences comprising a first single-stranded polynucleotide sequence and a second single-stranded polynucleotide sequence, and comprising a first portion and a second portion, ii) first primer (such as a first forward primer) comprising a first sequence that is complementary to the first portion of the first single-stranded polynucleotide sequence (such as the sense strand of a first portion of the target polynucleotide sequences), the first sequence is modified by having an insertion of a director, iii) second primer (such as a first reverse primer) comprising a second sequence that is complementary to the second portion of the second single-stranded polynucleotide sequence (such as the antisense strand of a second portion of the target polynucleotide sequences), the second sequence is modified by having an insertion of a complement of the second director, wherein the director is not complementary either to the first portion of the first single-stranded polynucleotide sequence or to the second portion of the second single-stranded polynucleotide sequence, iv) third primer (such as a second forward primer) fused at its 3′ end to a first driver, and v) fourth primer (such as second reverse primer) fused at its 3′ end to a second driver, wherein one of the first driver and the second driver has the same sequence as the director, and the other of the first driver and the second driver is the same sequence as the second director, wherein the 5′ end of either the first driver or the second driver is fused to a universal adapter sequence, and wherein the contacting step is under conditions sufficient for hybridizing the director either with the first driver or with the complement of the second driver, and for amplifying the plurality of target polynucleotide sequences to produce a) a first plurality of amplicons comprising a first single-stranded amplicon that comprises i) the first single-stranded polynucleotide sequence (such as a sense strand) having an insertion of the first director in either its 3′ or 5′ terminal regions and an insertion of the complement of the second director in its other terminal region, or ii) the second single-stranded polynucleotide sequence (such as an antisense strand) having an insertion of the second director in either its 3′ or 5′ terminal regions and an insertion of the complement of the first director in its other terminal region, and b) a second plurality of amplicons comprising a second single-stranded amplicon having the universal adapter sequence fused to its 5′ end, and comprising at its 3′ end either i) the first single-stranded polynucleotide sequence (such as a sense strand) containing an insertion of the first director in either its 3′ or 5′ terminal regions, and containing an insertion of the complement of the second director in its other terminal region, or ii) the second single-stranded polynucleotide sequence (such as an antisense strand) containing an insertion of the second director in either its 3′ or 5′ terminal regions, and containing an insertion of the complement of the first director in its other terminal region.

In one embodiment, it may be desirable to carry out the contacting steps c) and e) in a single reaction mixture.

In some embodiments, where it may be desirable to amplify a sense strand of double-stranded target polynucleotide sequence, the first primer and the third primer are forward primers, and the second primer and the fourth primer are reverse primers (FIGS. 1, 2, and 3B).

In some embodiments, where it may be desirable to amplify an antisense strand of double-stranded target polynucleotide sequence, the first primer and the third primer are reverse primers, and the second primer and the fourth primer are forward primers.

In one embodiment, the invention's method generates a second single-stranded amplicon that contains, fused in operable combination from the 5′ end to the 3′ end, a) the universal adapter sequence, and b) the first single-stranded polynucleotide sequence (such as sense strand) having an insertion of the first director in either its 3′ or 5′ terminal regions and an insertion of the complement of the second director in its other terminal region, or the second single-stranded polynucleotide sequence (such as antisense strand) having an insertion of the second director in either its 3′ or 5′ terminal regions and an insertion of the complement of the first director in its other terminal region.

In a particular embodiment, where it is desirable to include an index sequence in the amplified sense strand, the 5′ end of the first driver of the third primer is fused to a complement of the unique index sequence, and the 5′ end of the second driver of the fourth primer is fused to the universal adapter. This produces a second single-stranded amplicon fused at its 3′ end to the unique index sequence, and fused at its 5′ end to the universal adapter sequence (FIG. 1). Thus, the second single-stranded amplicon contains an insertion in its 3′ terminal region of a sense strand of the unique index sequence, and an insertion in its 5′ terminal region of a sense strand of the universal adapter sequence. In a particular embodiment, the second single-stranded amplicon contains, fused in operable combination from the 5′ end to the 3′ end, a) the universal adapter sequence, b) the first single-stranded polynucleotide sequence having an insertion of the director in either its 3′ or 5′ terminal regions and an insertion of the complement of the director in its other terminal region, or the second single-stranded polynucleotide sequence having an insertion of the director in either its 3′ or 5′ terminal regions and an insertion of the complement of the director in its other terminal region, and c) the unique index sequence. In a particular embodiment, the unique index sequence is comprised in an index adapter.

In a particular embodiment, where it is desirable to include an index sequence in the amplified antisense strand, the 5′ end of the first driver of the third primer is fused to the complement of the universal adapter sequence, and the 5′ end of the second driver of the fourth primer is fused to a unique index sequence. In one embodiment, the second single-stranded amplicon is fused at its 5′ end to the unique index sequence, and fused at its 3′ end to the universal adapter sequence. In a particular embodiment, the second single-stranded amplicon contains an insertion in its 5′ terminal region of a sense strand of the unique index sequence, and an insertion in its 3′ terminal region of a sense strand of the universal adapter sequence. In a particular embodiment, the unique index sequence is comprised in an index adapter. The invention's methods are exemplified by amplification of a sense strand of a target polynucleotide (FIG. 1). Thus, in one embodiment, the invention provides a method for amplifying a sense strand of a target polynucleotide sequences, comprising, contacting i) a sample comprising a plurality of target polynucleotide sequences, ii) first forward primer comprising a first sequence that is complementary to the antisense strand of a first portion of the target polynucleotide sequences, the first sequence is modified by having an insertion of a director, iii) first reverse primer comprising a second sequence that is complementary to the sense strand of a second portion of the target polynucleotide sequences, the second sequence is modified by having an insertion of a second director, neither director is complementary either to the first portion or to the second portion of the target polynucleotide sequences, iv) second forward primer fused at its 3′ end to a first driver, v) second reverse primer fused at its 3′ end to a second driver, wherein the first driver has the same sequence as the director, and the second driver has the same sequence as the second director, wherein the 5′ end of either the first driver or the second driver is fused to a universal adapter sequence, and wherein the contacting is under conditions sufficient for a) hybridizing the plurality of target polynucleotide sequences with the first forward primer and the first reverse primer, b) amplifying the plurality of target polynucleotide sequences to produce a first plurality of amplicons comprising a first sense strand amplicon comprising the target polynucleotide sequences having an insertion of the forward strand director in its 5′ terminal region and having an insertion of complement to the reverse strand director in its 3′ terminal region, c) contacting the second forward primer with the first sense strand amplicon, d) hybridizing the reverse strand driver with the complement of the reverse strand director of the first sense strand amplicon, e) contacting the second reverse primer with the first antisense strand amplicon, f) hybridizing the forward strand driver with the complement to the forward strand director of the first antisense strand amplicon, and g) amplifying the first sense strand amplicon and the first antisense strand amplicon to produce a second plurality of amplicons comprising a single-stranded amplicon that contains, fused in operable combination from the 5′ end to the 3′ end, the sense strand universal adapter sequence, and the target polynucleotide sequence having an insertion of the forward strand director in its 5′ terminal region and having an insertion of the complement to the reverse strand director in its 3′ terminal region.

In one embodiment, it may be desirable to carry out the two amplification steps in a single reaction mixture.

In a particular embodiment, where it is desirable to include an index sequence in the amplified sense strand, the second reverse primer comprises a complement to a unique index sequence fused at its 3′ end to the driver, and wherein the single-stranded amplicon contains at its 3′ end a sense strand of the unique index sequence. In a particular embodiment, the unique index sequence is comprised in an index adapter.

The invention further provides reaction mixtures for amplifying a double-stranded target polynucleotide sequence that contain a first single-stranded polynucleotide sequence and a second single-stranded polynucleotide sequence, and contains a first portion and a second portion, the reaction mixture comprising a) first primer (such as first forward primer) comprising a first sequence that is complementary to the first portion of the first single-stranded polynucleotide sequence (such as the antisense strand of a first portion of the target polynucleotide sequences), the first sequence is modified by having an insertion of a director, b) second primer (such as first reverse primer) comprising a second sequence that is complementary to the second portion of the second single-stranded polynucleotide sequence (such as the sense strand of a second portion of the target polynucleotide sequences), the second sequence is modified by having an insertion of a second director, wherein neither director is complementary either to the first portion of the first single-stranded polynucleotide sequence or to the second portion of the second single-stranded polynucleotide sequence, c) third primer fused at its 3′ end to a first driver, and d) fourth primer fused at its 3′ end to a second driver, wherein the first driver has the same sequence as the first director, and the second driver has the same sequence as the second director.

In a particular embodiment, where it is desirable to include an index sequence or a universal adapter sequence in the amplified sense strand, the reaction mixture comprises one or both of the 5′ end of the first driver of the third primer is fused to a unique index sequence, and the 5′ end of the second driver of the fourth primer is fused to the universal adapter sequence.

In a further embodiment where it is desired to amplify the sense strand of a target polynucleotide sequence, the 5′ end of the second driver of the fourth primer is fused to the universal adapter sequence. Optionally, the 5′ end of the first driver of the third primer is fused to a unique index sequence.

In a further embodiment where it is desired to amplify the antisense strand of a target polynucleotide sequence, the 5′ end of the first driver of the third primer is fused to the universal adapter sequence. Optionally, the 5′ end of the second driver of the fourth primer is fused to a unique index sequence.

The invention further provides kits for amplifying double-stranded target polynucleotide sequence, the kits comprising any one or more of the reaction mixtures herein.

### EXPERIMENTAL

The following examples serve to illustrate certain preferred embodiments and aspects of the present invention and are not to be construed as limiting the scope thereof.

### Example 1

### Initial Preliminary Method

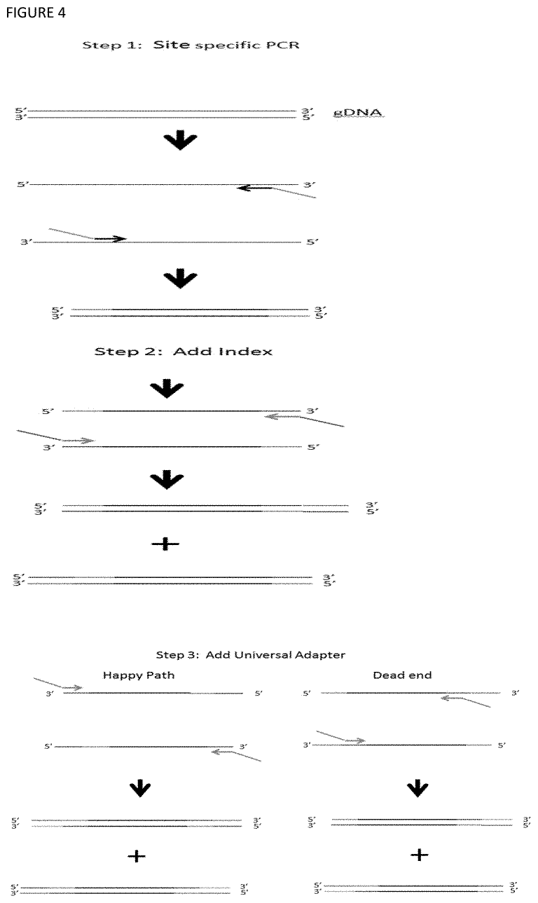

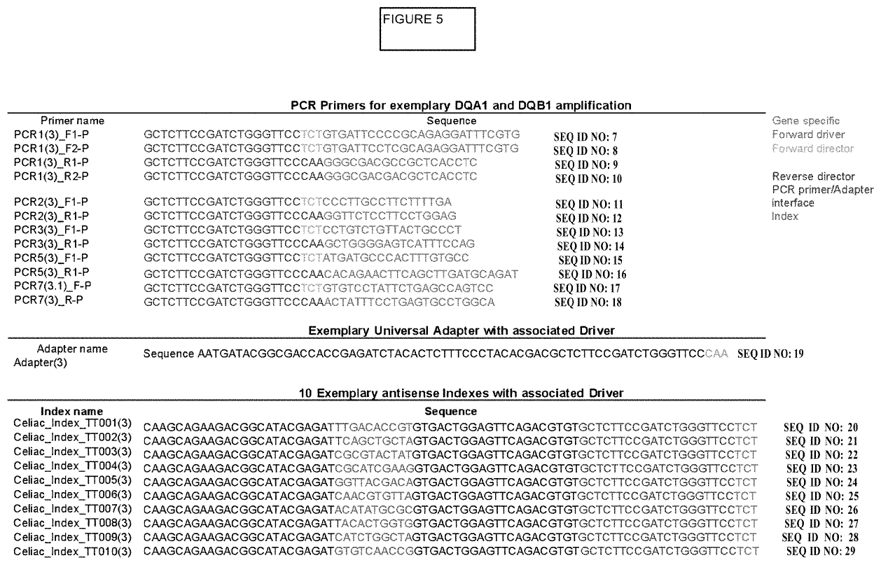

One initial strategy in developing the invention's methods (Example 1) attempted to sequence a few targeted regions of <300 bp of the hypervariable regions that define specific alleles in the celiac disease HLA DQA and HLA DQB genes, by using universal tags to randomly add the index adapter or universal adapter to either the forward or reverse strand. A schematic of the initial strategy is shown in FIG. 4.

As can be seen, because of the random nature of universal adapter and index adapter extensions, there were several undesired products generated by this method. Additionally, while much simpler than other library preparation methods, there are still four separate steps. Furthermore, presumably due to the number of product species a qPCR using the KAPA kit showed that the amount of sequenceable product was very limited. Also, as PCR optimization was performed we were unable to find sites within DQB1 that were both specific to DQB1 as well as not having polymorphisms that would lead to allelic dropout. Even the use of multiple primers for each target that included degenerate bases specific to each possible known variant at a given base continued to lead to allelic dropout.

### Example 2

### Sequence Amplification Using an Exemplary 3 Bp Driver Sequence

In view of the lack of optimization of the initial strategy to sequence targeted regions of the hypervariable regions that define specific alleles in the celiac disease HLA DQA and HLA DQB genes, alternative methods were carried out, in which the specificity requirement was lifted. This allowed for multiple genes such as HLA-DQB2 to be amplified as long as it reduced allelic dropout compared to the initial strategy of Example 1. In view of the removal of the specificity requirement, this new method therefore uses bioinformatics to eliminate the off-target reads. To resolve all of the non-sequenceable products, to reduce the number of steps, and to increase the efficiency of the reaction we modified the universal tags by adding a 3 bp change to the PCR primers and the 3′ end of the universal adapter and index primers. This now forces directional specificity to the universal adapter and index adapter incorporation. The invention's method is exemplified by the amplification of the sense strand of the target polynucleotide sequence as shown in FIGS. 1, 2, and 3B. As shown in FIG. 1, the final product of the invention's methods is a one sequenceable strand (either sense or antisense).

This invention's method can be performed in one step where the PCR primers, index sequences, and universal adapter sequences are all added into the same reaction mixture, substantially simultaneously. This reduces tech time, reduces the chances for transcription errors, and reduces the chances for secondary PCR contamination.

While there are technically four species present, two of these are precursor molecules to the final product. We have carried out the invention's methods to drive the reaction to the final product by using ⅙^{th} the PCR primer concentration compared to index adapter and universal adapter concentration and by using 50 cycles to exhaust reagents and drive to completion. Quantitation by KAPA kit confirmed that the amount of sequenceable product was high, with the last library generating 473 nM product. In some embodiments, it may be desired to reduce this concentration, for example by changing the PCR primer concentrations and/or number of PCR cycles. However, by guaranteeing all reactions have gone beyond the log-linear amplification and into the plateau phase the need for normalization should be reduced or even eliminated.

A) Design of Index Adapter and Universal Adapter

The index adapter and universal adapter were designed using methods known in the art (Illumina TruSeq Adapters Demystified Rev. A, © 2011 Tufts University Core Facility) as follows: (SEQ ID NOS: 3, 4, 5, 6):

LEFT OF INSERT
5 AATGATACGGCGACCACCGAGATCTACACTCTTTCCCTACACGAC--GC
TCTTCCGATC*T 3
3 GTTCGTCTTCTGCCGTATGCTCTA(INDEX)CACTGACCTCAAGTCTGC
ACA--CGAGAAGGCTAG*P 5
RIGHT OF INSERT
5 P*GATCGGAAGAGC--ACACGTCTGAACTCCAGTCAC(INDEX)ATCTC
GTATGCCGTCTTCTGCTTG 3
3 T*CTAGCCTTCTCG--CAGCACATCCCTTTCTCACATCTAGAGCCACCA
GCGGCATAGTAA 5

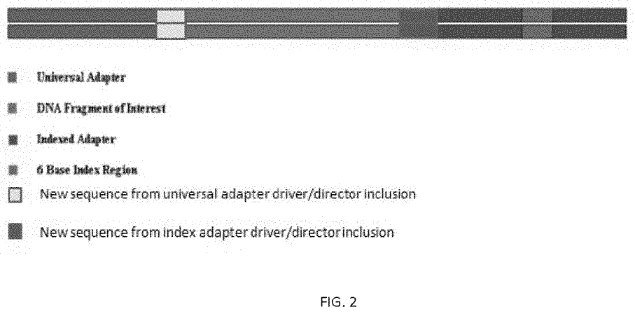

Paying attention only to the sense 5′ to 3′ strands in the image above, the region “left of insert” is provided in the invention's method by the forward primer that incorporates a universal primer that the Illumina adapter extends from in the sense orientation. The “right of insert” region referred to in the above image is provided by a reverse primer incorporating a universal primer that the index adapter extends from in the anti-sense orientation. During amplification, the sense orientation is transcribed from the antisense template, providing the completed upper strand. As showing in FIG. 2, the sequenceable sense strand (i.e., top strand) reads from the 5′ to 3′ end as follows: universal adapter, driver, target polynucleotide sequence, driver, and index adapter containing an exemplary 6-base index region.

Because the index is oriented in a 5′ to 3′ direction away from the insert, it cannot be directly incorporated by extension but instead is indirectly added by the extension of the sense strand into the index primer. As a result, the extended product is the reverse complement of the index primer. This means the index sequence reported to the MiSeq is the reverse complement of the index sequence contained in the index primer.

B) Exemplary Amplification of a Target Sense Strand

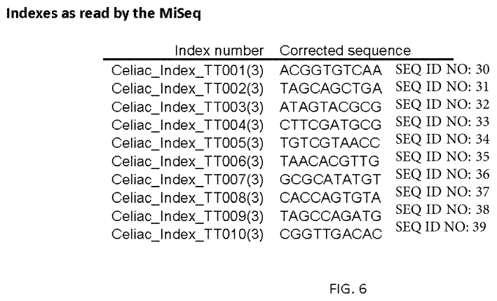

Sequences used in the exemplary amplification are shown in FIG. 5. The index sequences listed in FIG. 5 are the reverse complement of the index as read by the Illumina instrument. When submitting to CGSL this needs to be reverse complement transformed to what is actually read (FIG. 6).

The overall method uses four DQB1 amplicons and two DQA1 amplicon, each interrogating hypervariable regions for low resolution typing. The following is a more detailed discussion of the methodology and data for PCR1, then a more general discussion of the remaining PCRs.
1. Dilute all primers to 25 μM in Te. All concentrations for primers are assumed to be at 25 μM.
2. Add equal volumes of each of 12 primers for six products to make the Multiplex Primer Mix. This Multiplex Primer Mix is good for many setups. In this case added 50 μL of each were added but this mix could be made to any volume. A total of 2 μL, is needed per sample so this mix as shown is good for 300 samples.
3. For each sample reaction add the following reagents of Table 2:

TABLE 2

| Reagent | Per sample |
|---|---|
| Multiplex primer | 2 |
| mix (from above) | |
| Adapter(3) | 1 |
| 5x buffer | 5 |
| Water | 14.15 |
| dNTP | 0.75 |
| HiFi | 0.1 |
| Reagents added/well | 23 |

4. To each reaction well add 1 μL of DNA and 1 μL of sample specific index.
5. Cycle as follows:
   98 degrees Celsius for 5 minutes
   50 cycles of: 98 degrees Celsius for 30 seconds, 63 degrees Celsius for 15 seconds, 72 degrees Celsius for 1 minute
   72 degrees for 10 minutes
6. Run on a gel to confirm amplification of products and lack of amplification of NTC. Note that the six products are very similar in size and will co-migrate unless given 1-2 hours to separate on a large gel. For general confirm of amplification 15 minutes is sufficient, though individual exons will not be discrete.
7. Pool 5 μL of each of the samples in the run. Calculate total volume of all samples. Note this volume can be reduced if many samples are performed simultaneously.
8. Add 2 volumes Ampure XP for the volume of all samples.
9. Wash 3× with 70% ethanol.
10. Resuspend in 50 μL water.
11. Perform KAPA qPCR to determine molarity of sequenceable product. For reference, one exemplary batch was at 473 nM.
12. Dilute target to 10 nM and submit to CGSL.
13. Dilute target with PhiX or other library. Though this has not been tested, it is highly likely that without being run with other targets the limited library of six targets the MiSeq will have a problem focusing on the limited clusters that light up for some bases at some positions. This can lead to the entire run failing and is colloquially referred to as “phasing”.

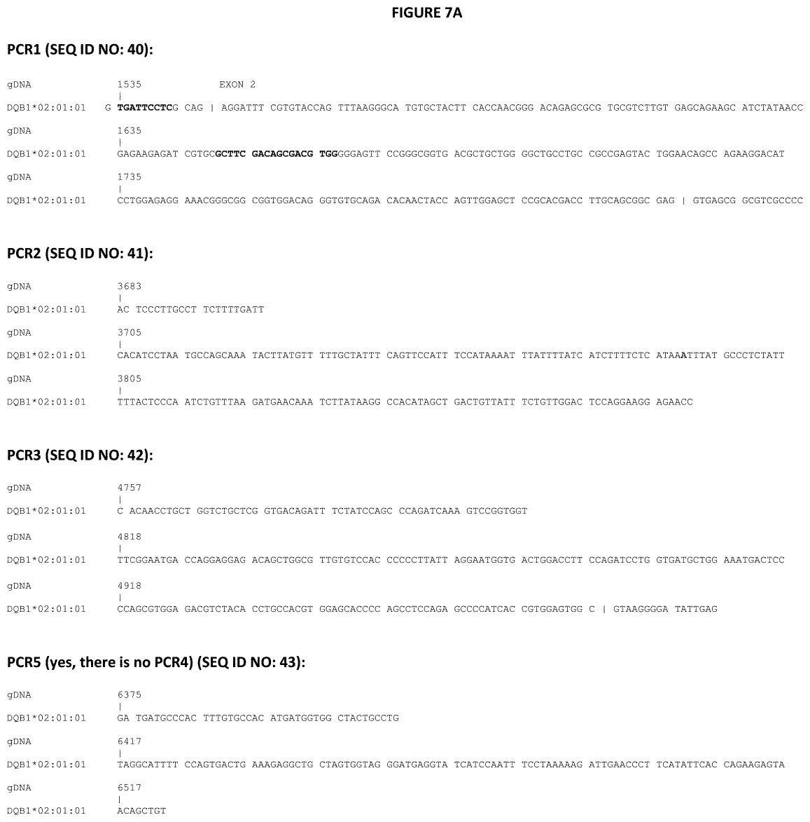

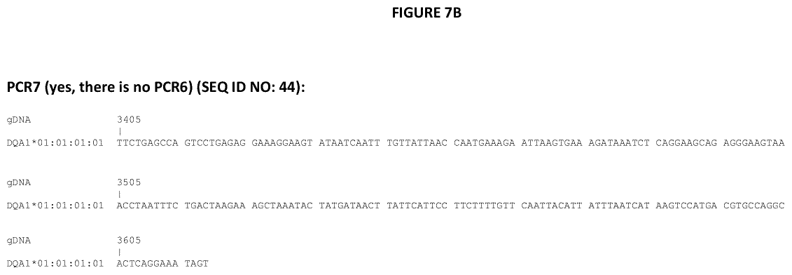

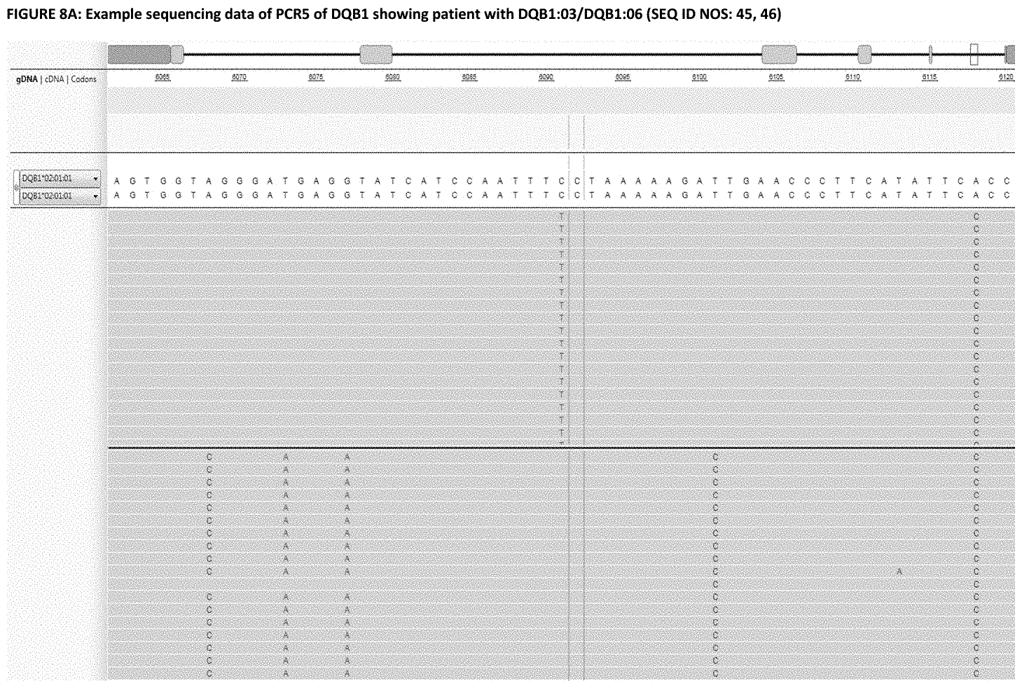

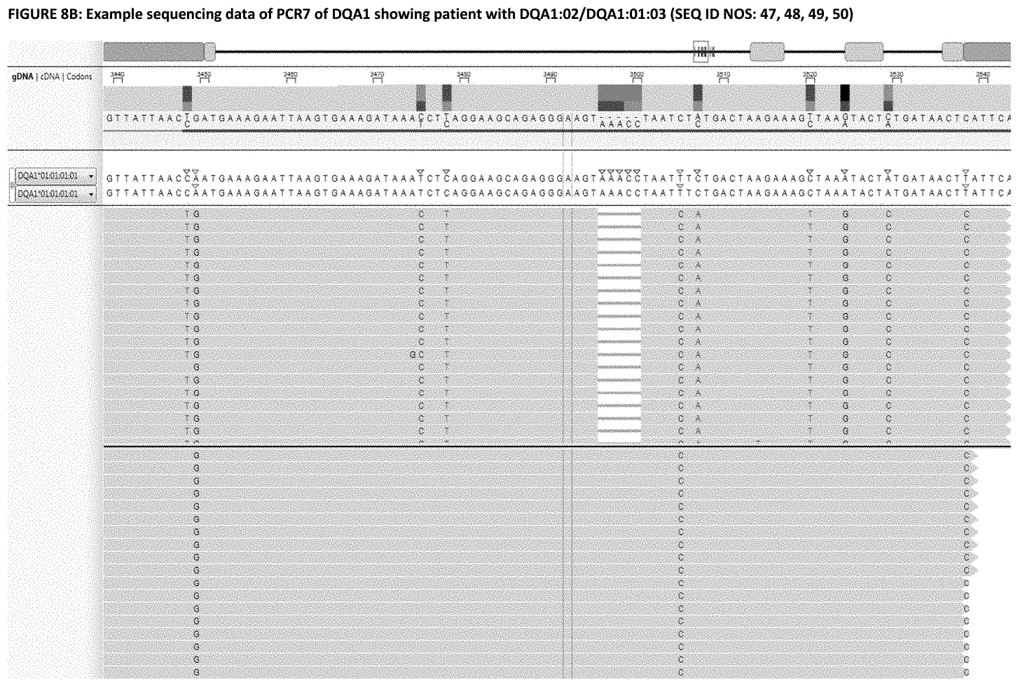

FIG. 7A-B shows region of genomic HLA-DQB1 and genomic HLA-DQA1 genes amplified by PCR, including initial PCR. Sequencing data shows a patient with DQB1:03/DQB1:06 (FIG. 8A) and another patient with DQA1:02/DQA1:01:03 (FIG. 8B).

### Example 3

### Effect of Driver Length on Percentage of Reads Using Exemplary 0, 1, 2, 3, 6, and 15 Bp Drivers

In Example 2, an exemplary three base pair (bp) driver sequence was successfully used to direct the index sequence and adapter sequence to their respective locations. This Example addresses whether other lengths of driver sequence may be used in the invention's methods.

To do this, six different driver sequence lengths were designed for each of the various PCRs, universal adapters, and index sequences including 0, 1, 2, 3, 6, and 15 bp. Sequences were selected to not interfere with either Illumina or gene specific regions, to avoid long stretches of the same base in a row, to have limited G/C ratios in order to not radically affect melting temperature, and were designed to not cause secondary structures such as hairpin folding. The sequences used are shown in FIG. 9

The PCR primers, index sequences, and universal adapter sequences were used as described above in Example 2. Five patients were used with each of 0, 1, 2, 3, 6, and 15 bp drivers. After the one step library preparation method was complete equal volumes of each sample were pooled, purified by Ampure, quantitated by qPCR (Kapa Library Quant Kit), diluted to 9 pM, diluted with PhiX, and sequenced on a 300 cycle MiSeq 300 kit.

The total number of reads associated with each PCR target region was determined using FastQC's Overrepresented Sequences tool. The total percent of reads each patient received in the flowcell are displayed in Table 3 and plotted in FIG. 10A-B.

TABLE 3
Percentage of flowcell reads associated with each driver length

| Driver bp | 0 bp | 1 bp | 2 bp | 3 bp | 6 bp | 15 bp |
|---|---|---|---|---|---|---|
| Sample 1 | 0.21 | 0.12 | 0.58 | 1.05 | 1.72 | 1.91 |
| Sample 2 | 0.04 | 0.33 | 0.92 | 1.12 | 1.53 | 1.66 |
| Sample 3 | 0.38 | 1.76 | 3.16 | 1.52 | 1.49 | 3.18 |
| Sample 4 | 0.13 | 0.21 | 3.56 | 0.97 | 0.96 | 2.52 |
| Sample 5 | 0.52 | 0.72 | 1.32 | 1.11 | 1.34 | 2.78 |
| Average | 0.25 | 0.63 | 1.91 | 1.15 | 1.41 | 2.41 |

FIG. 10A-B and Table 3 show that each of the exemplary 1, 2, 3, 6, and 15 bp drivers successfully produced total reads. Not using a driver (i.e., 0 bp driver) produced only a few targets of interest and had the lowest efficiency (i.e., lowest percentage of reads). The efficiency increased significantly when driver lengths of 1, 2, 3, 6, and 15 bp were included. For example Table 3 shows an increase in efficiency of about 27 fold (i.e. 2,700%) increase in efficiency from 0.13 percentage of total reads in the absence of a driver, to 3.56 percentage of total reads in the presence of a driver.

FIG. 10A-B and Table 3 also show that the general trend is for longer driver lengths to have higher efficiency as demonstrated by higher percentage of total reads. This demonstrates that in contrast to the invention's methods, not using a driver creates many non-sequenceable species, while including a driver length of at least 1 bp increases the efficiency.

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. A method for amplifying a target polynucleotide sequence, comprising
A) contacting a double-stranded target polynucleotide sequence with
i) a first primer modified by having an insertion of a first director, and
ii) a second primer modified by having an insertion of a second director, said contacting is under conditions sufficient for amplifying said double-stranded target polynucleotide sequence to produce a first plurality of amplicons comprising a first single-stranded amplicon that comprises at least one single strand of said double-stranded target polynucleotide sequence, said at least one single strand having an insertion of said first director in either its 3′ or 5′ terminal regions and an insertion of said second director in its other terminal region, and
B) contacting said at least one single strand with
i) a third primer fused at its 3′ end to a first driver, and
ii) a fourth primer fused at its 3′ end to a second driver,
wherein one of said first driver has the same sequence as said first director, and the said second driver has the same sequence as said second director, and
wherein said contacting is under conditions sufficient for amplifying said at least one single strand produced in step A) to produce a second plurality of amplicons comprising at its 3′ end said at least one single strand containing an insertion of said first director in either its 3′ or 5′ terminal regions, and containing an insertion of said complement of said second director in its other terminal region.

2. The method of claim 1, wherein the 5′ end of said first driver is fused to a universal adapter, or the 5′ end of said second driver is fused to a complement of said universal adapter sequence, and wherein said second single-stranded amplicon comprises said universal adapter sequence fused to its 5′ end.

3. The method of claim 1, wherein said contacting of steps A) and B) is in a single reaction mixture.

4. The method of claim 1, wherein said first primer and said third primer are forward primers, and said second primer and said fourth primer are reverse primers.

5. The method of claim 1, wherein said first primer and said third primer are reverse primers, and said second primer and said fourth primer are forward primers.

6. The method of claim 2, wherein said second single-stranded amplicon contains, fused in operable combination from the 5′ end to the 3′ end,
a) said universal adapter sequence, and
b) said at least one single strand having an insertion of said first director in either its 3′ or 5′ terminal regions and an insertion of said complement of said second director in its other terminal region.

7. The method of claim 2, wherein the 5′ end of said first driver of said third primer is fused to the complement of a unique index sequence and the 5′ end of said second driver of said fourth primer is fused to said universal adapter sequence.

8. The method of claim 7, wherein said second single-stranded amplicon is fused at its 3′ end to said unique index sequence, and fused at its 5′ end to said universal adapter sequence.

9. The method of claim 8, wherein said second single-stranded amplicon contains an insertion in its 5′ terminal region of a sense strand of said unique index sequence, and an insertion in its 3′ terminal region of a sense strand of said universal adapter sequence.

10. The method of claim 2, wherein the 5′ end of said first driver of said third primer is fused to the complement of said universal adapter sequence and the 5′ end of said second driver of said fourth primer is fused to a unique index sequence.

11. The method of claim 10, wherein said second single-stranded amplicon is fused at its 5′ end to said unique index sequence, and fused at its 3′ end to said universal adapter sequence.

12. The method of claim 11, wherein said second single-stranded amplicon contains an insertion in its 5′ terminal region of a sense strand of said unique index sequence, and an insertion in its 3′ terminal region of a sense strand of said universal adapter sequence.

13. The method of claim 1, further comprising sequencing said single-stranded amplicon comprised in said second plurality of amplicons.

14. The method of claim 1, wherein said conditions in steps A) and B) are sufficient to produce said second single-stranded amplicon at a higher efficiency than in the absence of one or more of said first director, said second director, said first driver, and said second driver.

15. The method of claim 1, wherein the concentration of one or both of said first primer and said first primer is 25% or less than the concentration of one or both of said second primer and said second primer.

16. The method of claim 1, wherein said amplifying of one or both of steps A) and B) comprises 50 or fewer PCR cycles.

17. The method of claim 1, wherein said target polynucleotide sequence comprises genomic DNA.

18. The method of claim 1, wherein said genomic DNA comprises a variable sequence of an allele.

19. The method of claim 1, wherein said second single-stranded amplicon is an amplified sense strand of said double-stranded target polynucleotide sequence or an amplified antisense strand of said double-stranded target polynucleotide sequence.

20. A method for amplifying a target polynucleotide sequence, comprising contacting
i) a sample comprising a plurality of double-stranded target polynucleotide sequences comprising a first single-stranded polynucleotide sequence and a second single-stranded polynucleotide sequence, and comprising a first portion and a second portion,
ii) first primer comprising a first sequence that is complementary to said first portion of said first single-stranded polynucleotide sequence, said first sequence is modified by having an insertion of a director, wherein said director is not complementary either to said first portion of said first single-stranded polynucleotide sequence or to said second portion of said second single-stranded polynucleotide sequence,
iii) second primer comprising a second sequence that is complementary to said second portion of said second single-stranded polynucleotide sequence, said second sequence is modified by having an insertion of a second director, wherein neither said first director nor said second director is complementary either to said first portion of said first single-stranded polynucleotide sequence or to said second portion of said second single-stranded polynucleotide sequence,
iv) third primer fused at its 3′ end to a first driver, and
v) fourth primer fused at its 3′ end to a second driver,
wherein said first driver has the same sequence as said first director, and said second driver is the same sequence as the second director, and
wherein said contacting is under conditions sufficient for hybridizing said first director with the complement of said first driver and said second director with the complement of said second driver, and for amplifying said plurality of target polynucleotide sequences to produce
a) a first plurality of amplicons comprising a first single-stranded amplicon that comprises
i) said first single-stranded polynucleotide sequence having an insertion of said first director in either its 3′ or 5′ terminal regions and an insertion of said complement of said second director in its other terminal region, or
ii) said second single-stranded polynucleotide sequence having an insertion of said second director in either its 3′ or 5′ terminal regions and an insertion of said complement of said first director in its other terminal region, and
b) a second plurality of amplicons comprising a second single-stranded amplicon having at its 3′ end either
i) said first single-stranded polynucleotide sequence containing an insertion of said first director in either its 3′ or 5′ terminal regions, and containing an insertion of said complement of said second director in its other terminal region, or
ii) said second single-stranded polynucleotide sequence containing an insertion of said second director in either its 3′ or 5′ terminal regions, and containing an insertion of said complement of said first director in its other terminal region.

21. The method of claim 20, wherein the 5′ end of either said first driver or said second driver is fused to a universal adapter sequence or to the complement of a universal adapter sequence.

22. The method of claim 20, wherein said second single-stranded amplicon is an amplified sense strand of said double-stranded target polynucleotide sequence or an amplified antisense strand of said double-stranded target polynucleotide sequence.

23. A method for amplifying a sense strand of a target polynucleotide sequence, comprising, contacting
i) a sample comprising a plurality of target polynucleotide sequences comprising sense and antisense strands,
ii) first forward primer comprising a first sequence that is complementary to the sense strand of a first portion of said target polynucleotide sequences, said first sequence is modified by having an insertion of a director, wherein said director is not complementary either to said first portion or to said second portion of said target polynucleotide sequences,
iii) first reverse primer comprising a second sequence that is complementary to the antisense strand of a second portion of said target polynucleotide sequences, said second sequence is modified by having an insertion of a second director, wherein said director is not complementary either to said first portion or to said second portion of said target polynucleotide sequences,
iv) second forward primer fused at its 3′ end to a first driver,
v) second reverse primer fused at its 3′ end to a second driver
wherein said first driver has the same sequence as said first director, and the said second driver has the same sequence as said second director, and
wherein said contacting is under conditions sufficient for
a) hybridizing said plurality of target polynucleotide sequences with the complement of said first forward primer and said first reverse primer,
b) amplifying said plurality of target polynucleotide sequences to produce a first plurality of amplicons comprising a first sense strand amplicon comprising said target polynucleotide sequences having an insertion of said forward strand director in its 5′ terminal region and having an insertion of complement to said reverse strand director in its 3′ terminal region,
c) contacting said second forward primer with said first sense strand amplicon,
d) hybridizing said reverse strand driver with the complement of said reverse strand director of said first sense strand amplicon,
e) contacting said second reverse primer with said first antisense strand amplicon,
f) hybridizing said forward strand driver with said complement to said forward strand director of said first antisense strand amplicon, and
g) amplifying said first sense strand amplicon and said first antisense strand amplicon to produce a second plurality of amplicons comprising a single-stranded amplicon that contains, fused in operable combination from the 5′ end to the 3′ end, said sense strand universal adapter sequence, and said target polynucleotide sequence having an insertion of said forward strand director in its 5′ terminal region and having an insertion of said complement to said reverse strand director in its 3′ terminal region.

24. The method of claim 23, wherein the 5′ end of either said first driver or said second driver is fused to a universal adapter sequence.

25. The method of claim 23, wherein said contacting of steps c) and e) is in a single reaction mixture.

26. The method of claim 23, wherein said second forward primer comprises a complement to a unique index sequence fused at its 3′ end to said driver, and wherein said single-stranded amplicon contains at its 3′ end a sense strand of said unique index sequence.

27. A reaction mixture for amplifying a double-stranded target polynucleotide sequence that contains a first single-stranded polynucleotide sequence and a second single-stranded polynucleotide sequence, and contains a first portion and a second portion, said reaction mixture comprising
a) first primer comprising a first sequence that is complementary to said first portion of said first single-stranded polynucleotide sequence, said first sequence is modified by having an insertion of a first director, wherein said director is not complementary either to said first portion of said first single-stranded polynucleotide sequence or to said second portion of said second single-stranded polynucleotide sequence,
b) second primer comprising a second sequence that is complementary to said second portion of said second single-stranded polynucleotide sequence, said second sequence is modified by having an insertion of a second director, wherein said second director is not complementary either to said first portion of said first single-stranded polynucleotide sequence or to said second portion of said second single-stranded polynucleotide sequence,
c) third primer fused at its 3′ end to a first driver, and
d) fourth primer fused at its 3′ end to a second driver,
wherein said first driver has the same sequence as said director, and the second driver is said the same sequence as the second director.

28. The reaction mixture of claim 27, said reaction mixture comprises said first sequence hybridized along a portion of its length to said first portion of said first single-stranded polynucleotide sequence, and said second sequence hybridized along a portion of its length to said second portion of said second single-stranded polynucleotide sequence.

29. The reaction mixture of claim 27, said reaction mixture comprises said first driver hybridized to the complement of said first director, and said second driver hybridized to said complement of said second director.

30. The reaction mixture of claim 27, wherein said reaction mixture comprises one or both of the 5′ end of said first driver of said third primer is fused to a unique index sequence, and the 5′ end of said second driver of said fourth primer is fused to the complement of said universal adapter sequence.

31. The reaction mixture of claim 27, wherein the 5′ end of said first driver of said third primer is fused to a unique index sequence.

31. The reaction mixture of claim 27, the 5′ end of said second driver of said fourth primer is fused to the complement of said universal adapter sequence.

33. The reaction mixture of claim 27, wherein the 5′ end of said first driver of said third primer is fused to said universal adapter sequence.

34. The reaction mixture of claim 27, the 5′ end of said second driver of said fourth primer is fused to the complement of a unique index sequence.

35. A kit for a amplifying double-stranded target polynucleotide sequence, said kit comprising the reaction mixture of claim 27.
